(19) United States
(12) Patent Application Publication
(10) Pub. No.: US 2008/0187605 A1
(43) Pub. Date: Aug. 7, 2008

(54) PREVENTING PATHOLOGICAL NERVE CELL SUICIDE (NEUROAPOPTOSIS) IN IMMATURE NERVOUS SYSTEMS

(76) Inventor: John W. Olney, Ladue, MO (US)

Correspondence Address: PATRICK D. KELLY, 11939 MANCHESTER #403, ST. LOUIS, MO, 63131, US

(21) Appl. No.: 11/670,960

(22) Filed: Feb. 2, 2007

(51) Int. Cl. A61K 33/00; A61K 31/05; A61K 31/136; A61K 31/4164; A61K 33/14; A61K 31/045; A61K 31/125; C01B 23/00; A61P 25/00; A61K 31/357; A61K 31/426; A61K 31/515; A61K 31/53; A61K 31/55

(52) U.S. Cl. 424/722; 424/600; 514/385; 514/647; 424/677; 424/718; 424/722; 514/221; 514/242; 514/270; 514/367; 514/454; 514/676; 514/731

(57) ABSTRACT

By disrupting a natural process used by developing mammalian brains to prune and delete surplus neurons, surgical anesthetics and other drugs that suppress brain activity in fetuses and infants can trigger permanent pathological brain damage. That type of damage can be prevented by drug interventions that block one or more “upstream” events that otherwise would lead to the release of “Cytochrome C”, a messenger molecule that triggers apoptosis (programmed cell death) among immature neurons. Lithium is a potent protective agent that can be coadministered along with ketamine or other NMDA-acting or GABA-acting anesthetics and anticonvulsants. Xenon gas triggers only mild damage, and can enable improved anesthesia when combined with other drugs. Other protective drugs (also called safener drugs), and treatments that can prevent or minimize fetal alcohol syndrome, also are disclosed.

### RELATED APPLICATION

This application claims the benefit and priority dates of Patent Cooperation Treaty application PCT/US2005/027460, filed Aug. 2, 2005 and published as WO 2006/017524, which claimed the benefit of U.S. provisional application 60/598,390, filed Aug. 2, 2004.

### BACKGROUND OF THE INVENTION

This invention relates to medicine, neurology, and anesthesiology, and especially pertains to obstetric and pediatric medicine, involving fetuses, infants, and pregnant mothers.

When various parts of the body are being formed during embryonic or fetal development, a surplus number of cells are initially provided for that part of the body; then, in the course of normal development, many of these cells are deleted, by a process called “apoptosis”, which immature organisms use to control growth, development, and cell numbers.

There is an ideal number of cells that make up any organ or limb, in the body of any human or other animal, during each of its various stages of growth, development, and maturity. For optimal health, the body tries to maintain cell numbers in each organ or limb at an ideal level, which changes over time as an organism grows and matures.

After an organism reaches maturity, the cells in nearly all types of tissues continually undergo a natural process of replacement, as they become aged and no longer vigorous. When an aging cell dies, a neighboring cell of the same kind (or in some cases a specialized precursor cell) undergoes cell division, to form a new cell to replace the one that died. This process normally operates in a balanced manner, so that cell numbers for each tissue or organ are maintained at an ideal level.

However, under some circumstances, the cell death process can exceed the rate of replacement. If this occurs and is sustained over time, the affected organ can become impaired or dysfunctional. For example, if the liver is infected by a viral hepatitis that kills large numbers of cells and prevents them from regenerating, the person can suffer serious liver damage, and may die unless he/she receives a liver transplant.

In the opposite direction, it is also possible for the rate of cell reproduction to escape its normal controls, and begin to outrun the rate of cell death. This condition usually results in cancers, benign tumors, or similar problems, and may also be involved in autoimmune disorders and other problems.

Thus, apoptosis, a process by which cells actively commit suicide, is essential for maintaining cell numbers at ideal levels, in the organs and connective tissues of an adult animal.

Apoptosis also plays crucially important roles in the development and growth of an embryo or fetus. When the body is being formed, during embryonic life, a surplus number of cells is provided for many parts of the body. Then, in the course of normal development, some of those cells are killed and deleted by apoptosis. For example, when a human hand or foot is being formed, it initially has the appearance of a webbed structure, like a duck's or frog's foot; then, by apoptotic death and deletion of the cells that make up the webbing, the extremity is shaped into normal and separate fingers and toes. In a similar manner, the valves in and around the heart are initially formed as simple tubes. Portions of those tubes then dissolve, through a process of apoptosis, leaving behind flaps with specific shapes and connections that make the valves functional.

Thus, controlled apoptosis plays a major role in normal development of various parts of the body. However, the extent to which apoptosis is involved in shaping an organ varies considerably from one organ to another, and for some organs, the role of apoptosis involves factors that remain unresolved mysteries.

Apoptosis plays a major role in the development and growth of the immature brain. In a developing mammalian brain, surplus and redundant neurons are initially created, then some of those neurons are killed, and deleted, by apoptotic mechanisms that are comparable to a gardener pruning unwanted shoots and branches from a plant.

Based on recent research, it is becoming clear that this natural process of programmed neuronal death, in immature brains, can be accelerated and increased by certain types of external factors, in ways that can inflict permanent pathological damage on an immature brain. For example, in a series of recent studies (reviewed in Olney et al 2004a), John Olney (the inventor herein) and his coworkers discovered that during “synaptogenesis” (i.e., a period in fetal and neonatal life when neurons are growing rapidly and establishing synaptic connections with one another), neurons are prone to commit suicide at abnormally high rates, if something interferes with the normal processes by which the neurons create, activate, and evaluate synaptic junctions with other developing neurons. That process is described in more detail below.

The period of synaptogenesis, also known as the brain growth spurt period, occurs in different species at different times, relative to the time of birth (Dobbings and Sands 1979). In rats and mice, it continues after birth, for roughly 3 weeks of neonatal life. In humans, it begins in the second trimester of pregnancy, and lasts until about 3 to 4 years after birth. Thus, data generated in postnatal rodents have relevance to both the prenatal (fetal) and postnatal (infant) periods in humans.

Factors that can cause immature neurons to commit pathological levels of suicide may be either environmental or genetic. For example, various drugs that suppress neuronal activity can trigger neuronal suicide, in immature rodent brains. These types of drugs usually impart anesthetic, sedating, anti-convulsant, or similar effects in both immature and adult brains. Exposure of neurons to such drugs in adult animals, even for many hours, will not cause the adult neurons to commit suicide; however, exposure of immature neurons to such drugs, even for only a few hours, can cause neuronal suicide. If humans are as sensitive as rodents to this neurotoxic effect, this would signify that treating pregnant women or human infants with such drugs poses risks of triggering neuronal suicide in the immature brain (treatment of pregnant females will create this type of risk, only if such drugs reach and enter the circulating blood in substantial quantities; administration by other modes, such as epidural anesthetics that enter spinal fluids only, are not likely to cause such problems, since they do not reach circulating blood in any substantial quantity, and will not cross the placental barrier and reach the fetus).

Alternately, various genetic mutations have been created and tested in mice, which cause certain neurotransmitter or neurotrophic substances to be dysfunctional in regulating neuronal activity. Since the proper construction, activation, and operation of neurons and synaptic activity require each and all of numerous different proteins and enzymes to function properly, the deletion of even a single gene that is involved in the process typically creates a “break in the chain”, which will hinder synaptogenesis. Regardless of which particular form or manifestation the hindrance may take, the simple fact that synaptogenesis is indeed being hindered, and suppressed, will cause neurons in the brain to commit suicide in abnormally large numbers, during the period of synaptogenesis.

When nerve cells die, they are not replaced. Neurons in most regions of the developing central nervous system (CNS) simply do not have the regenerative capacity to form new nerve cells, to replace neighboring cells that have died. Once a neuron dies, it is deleted from the nervous system forever, and not replaced.

As a result, if an increased rate of apoptotic cell death occurs in the immature brain, it will have a much more lasting and profound impact on an animal, than a temporary increase in apoptotic cell death in other tissues of the body. If neurons are deleted in abnormally large numbers from an individual's brain, during fetal development, it will permanently impair the ability of that individual to function normally, and the extent and severity of the impairment will be generally proportional to the numbers of neurons that were abnormally deleted, during that developmental stage in the immature brain.

In the central nervous system (CNS, i.e., the brain, spinal cord, and retina), the role of apoptosis in normal development is poorly understood. It is known that during normal development of the CNS, a certain percentage of neurons will die by apoptosis; however, scientists have not had reliable methods for detecting neuro-apoptosis, or for measuring the varying rates at which it occurs, in various regions of the developing CNS. As will be described below, Olney and colleagues, using methods that have only recently become available, have made important new inroads in understanding the role of apoptosis in normal development, and they recently have discovered ways in which the rate of neuro-apoptosis can become abnormally increased, thereby causing excessive and pathological deletions of neurons from immature brains.

Before describing the recent findings of Olney and colleagues, it is necessary to explain that there is a great deal of confusion in the medical literature, regarding the role of apoptosis in diseases of the CNS. In the human CNS, very early during development, progenitor cells divide and multiply and their progeny differentiate into neurons. Once that has happened, the process of neuronal generation stops and, with very minor exceptions, there are no additional neurons formed in the brain for the remainder of a person's life.

Because neurons in the mature brain do not have the capacity to generate new neurons, they also do not have a pathological tendency to cause cancer. It is true that cancer sometimes occurs in the brain or spinal tissue; however, except in very rare cases, these are not tumors of nerve cells; they are tumors of glial cells, which provide a supportive matrix in the brain. Glial cells cannot send or receive nerve signals; however, they do have regenerative potential, and therefore, they are susceptible to forming cancer.

Mature neurons do not divide and multiply their numbers, so there is no provision among neuronal cells for an apoptotic process to keep neuronal multiplication in check. In the literature, there has been much speculation that apoptosis may play a role in various neurodegenerative diseases of the adult CNS; however, evidence to support that speculation is not very strong. Therefore, at the present time, it is uncertain whether apoptosis is a cell death mechanism that plays a significant role in any neurodegenerative diseases of the adult CNS.

Another factor that has also contributed to confusion, misinterpretation, and misunderstanding in the medical literature centers on the relationship between two very different processes by which nerve cells die in the developing CNS. Those two very different processes are apoptotic cell death, and excitotoxic cell death.

As already mentioned, apoptotic nerve cell death occurs naturally, to some extent, in the developing CNS, as part of the two-part CNS procedure that involves (i) initially creating an oversupply of neurons, and then (ii) deleting neurons that appear to be surplus, as evidenced by a lack of activity and interactions with other neurons. Accordingly, during synaptogenesis, neuron-apoptosis can be triggered by abnormally low activity, rather than abnormally high activity.

By contrast, excitotoxic nerve cell death does not occur as a natural phenomenon during development, and it is not triggered by abnormally low levels of neuronal activity. Instead, as suggested by the name, excitotoxic neuronal death is caused when an excitatory neurotransmitter (glutamate) creates such abnormally high levels of neuronal activity that neurons become exhausted and depleted to a point where they begin dying. The cellular and physiological factors that cause neurons to begin dying, when they are severely overstimulated by glutamate, are reviewed elsewhere (e.g., Olney and Ishimaru 1999, and U.S. Pat. No. 5,767,130 (Olney 1998)), so that information is not repeated herein.

In immature brains, excitotoxic cell death most commonly is triggered by hypoxia and/or ischemia (i.e., inadequate oxygen and/or blood supply, sometimes referred to as perinatal asphyxia, which can occur during birth if the umbilical cord becomes wrapped tightly around the neck, or if the mother suffers from shock, severe blood loss, or other problems). It can also be caused by hypoglycemia, status epilepticus, traumatic brain injury, and various other types of medical problems or crises.

A major reason for the confusion between apoptotic neuronal death, versus excitotoxic neuronal death, is that in immature brains, certain types of conditions (including hypoxia/ischemia, hypoglycemia, status epilepticus, and traumatic brain injury) can cause both types of neuronal death, in a sequence in which a first wave of excitotoxic neuronal death is followed by a subsequent wave of apoptotic neuronal death. Accordingly, most researchers have not used methods that can differentiate between those two processes, and they have not adequately recognized the time-dependent sequence of excitotoxic damage, followed by apoptotic damage. Rather, they have tended to lump together those two very different types of neuronal death (which can be triggered by a single event), as if they were a single combined process. Some researchers even use terms such as, “excitotoxic apoptosis”, a misnomer that reflects the confused state of affairs in this area of research.

Olney and colleagues have described methods and criteria for distinguishing between excitotoxic versus apoptotic cell death in immature brains, and they have clarified the relationship between the two processes (e.g., Ishimaru et al 1999, Olney 2003, and Young et al 2004, and Bayly et al 2006). For example, they have shown that in immature rat brains, either hypoxia/ischemia or head trauma can trigger both an acute type of excitotoxic neuronal death (which is completed within 4 hours), and a delayed type of apoptotic cell death (which evolves over 8 to 24 hours). They also have shown that those two very different processes can be distinguished quite readily, not just because they occur at separate locations and at different times, but also by using tools such as ultrastructural evaluation, which clearly show that the types and sequences of morphological damage and alteration that characterize excitotoxic damage are clearly different from the events and results that characterize apoptosis.

It is important to recognize and understand the distinctive characteristics of those two different types of neuronal death processes, because it is very likely that prophylactic and treatment methods that can prevent or minimize and control one type of cell death, will be ineffective in preventing or reducing the other type of cell death. Indeed, because they involve diametrically-opposed triggering events (i.e., inadequate neuronal activity leads to apoptosis in immature brains, while excessive activity leads to excitotoxic brain damage), there is a potential risk that a treatment designed to prevent one type of damage will end up increasing the other type of damage (indeed, that type of effect was apparently confirmed by research by the inventor herein, described below).

Just as importantly, since the apoptotic cell death process has a delayed and more protracted time course, physicians and researchers need to understand that there may be, in at least some apoptotic cell death processes, a longer window of opportunity for therapeutic intervention than is the case for excitotoxic cell death. This has also been shown by research by the inventor herein, as described below.

To explain further the concept of deprivation leading to apoptosis, it is instructive to examine what happens in head trauma. A concussive blow creates a severe impact, at a certain place on the skull. Inside the skull, immediately adjacent to the impact, an area of excitotoxic neurodegeneration will occur, acutely and rapidly, leading to end-stage cell death within about 2 to 4 hours.

Subsequently, beginning at about 8 hours, signs of apoptotic cell death will begin to emerge, often at sites that are remote from the brain regions directly beneath the skull at the point of impact. This apoptotic cell death process evolves, with different degrees of intensity in various brain regions, over a period of approximately 8 to 24 hrs.

The extent, and the patterns, of apoptotic neuronal death that will occur, at brain regions that are remote from the location of the direct brain tissue injury, have been discovered, by Olney and colleagues, to depend heavily on whether the immature brain is passing through a developmental stage called “synaptogenesis” (also known as the “brain growth spurt” period). During this developmental period of synaptogenesis, all neurons in the brain are receiving synaptic inputs from distant neurons, and they also are sending out long fibers to make synaptic contacts with distant neurons. The initial traumatic event triggers a wave of excitotoxicity, which will kill nerve cells that are “subjacent” to the impact site. A type of semi-liquid “wave” that travels through the brain tissue can also cause physical strain and sheering forces, which can injure and even sever some of the axonal fibers through which neurons in one brain region make synaptic contacts with neurons in other regions. As a result, various neurons that are remote from the direct impact site will be deprived of incoming synaptic signals, and/or they will be deprived of synaptic targets. Accordingly, if excitotoxic destruction of neurons, and physical destruction of fiber connections, causes neurons in other brain regions to be deprived of both synaptic inputs, and synaptic targets, the remote neurons will sense that they are failing to perform their synaptic mission. That failure will trigger an apoptotic response, in which the remote neurons will commit suicide.

As will be discussed in more detail below, various drugs that can inhibit neuronal activity can also cause neurons in an immature brain to be placed in an “activity-deprived” condition, even when the brain has suffered no physical or mechanical damage. Recent research has shown that developing neurons in an immature brain require a certain level of nerve signal activity, in order to meet what can be regarded as a “synaptic connectivity schedule”. If neurons in an immature brain are suppressed by such drugs, to abnormally low levels of activity, they will not meet their schedules. If they sense that they are failing to meet their schedule, they interpret that as an indicator that they apparently belong in the set of neurons that simply failed to be chosen and woven into the interactive “fabric” of brain activity. As a direct result of that lack of activity, certain proteins will activate certain signals, and those neurons will be killed, by the same process of apoptosis that is actively pruning out and eliminating other unwanted and unused neurons in a process that, in a healthy brain, brings the surplus number of starting neurons down to a proper long-term level.

Apoptosis is recently becoming recognized as a disease mechanism in the developing nervous system. In contrast to the uncertain situation in adult neurodegenerative diseases, it is clear from the research data, and from the conclusions that are supported by those data, that apoptosis decidedly plays an important and even major role in a number of entirely natural diseases that afflict immature brains and spinal cords. It also is becoming clear that apoptosis, arising from nerve signal deprivation caused by a pregnant female's intake of ethanol (i.e., ethyl alcohol, the intoxicant in beer, wine, and liquor) is the major causative factor in the type of brain damage known as “fetal alcohol syndrome”.

In addition, very recent research has created a growing body of evidence that apoptotic damage to immature brains can be caused by “iatrogenic” situations, in which the immature brain is exposed, during medical treatments for other conditions, to drugs that can trigger neuroapoptosis.

The data and conclusion that certain types of drugs that are commonly used as sedatives, anesthetics or anticonvulsants in pediatric and obstetric medicine, can trigger developmental neuroapoptosis, are only very recently becoming recognized. Olney et al. recently have shown that exposure of the immature CNS to any of several classes of pharmacological agents, during the rapid brain growth period of synaptogenesis, causes developing neurons in the brain, spinal cord, and retina to commit suicide (i.e., to die by apoptosis). Drugs that will trigger neuroapoptosis in these fetal and neonatal animal models include three major categories of compounds, all of which have various known effects and uses. Those three categories are:

(1) drugs that block receptors for an important excitatory transmitter system, known as the “NMDA” subclass of glutamate receptor (NMDA refers to N-methyl-D-aspartate, a probe drug that was discovered to powerfully and selectively activate those particular receptors); stimulation of NMDA receptors by the excitatory transmitter, glutamate, increases neuronal activity, and drugs that block these receptors (known as NMDA antagonist drugs) suppress neuronal activity.

(2) drugs that activate receptors for an important inhibitory transmitter system, known as the GABAA receptor system (GABA refers to gamma-amino-butyric acid, which is the predominant inhibitory transmitter in the mammalian brain. Activation of GABA inhibitory receptors suppresses neuronal activity (compare with blockade of NMDA excitatory receptors, which also suppresses neuronal activity).

(3) drugs that block “voltage-gated” sodium ion channels, which are crucial in allowing neurons to undergo the ionic flows and chemical surges that create firing events and signal transmissions. Blocking these sodium ion channels also suppresses neuronal activity.

It also should be noted that ethanol, the culprit in fetal alcohol syndrome, strongly suppresses nerve signal transmissions, by two separate mechanisms: (i) it blocks NMDA excitatory receptors; and, (ii) it activates GABA-A inhibitory receptors.

These neuro-apoptosis findings from in vivo animal tests, which have been recently published by Olney et al. (e.g., Olney et al 2004a and 2004b, and Jevtovic-Todorovic et al 2003), have considerable significance in a public health context, and these reports have generated substantial controversy in the medical community, because a number of drugs that are commonly used in obstetric and pediatric medicine (mainly as sedatives, anxiolytics, anticonvulsants, or anesthetics) fall squarely into the categories listed above. The status of this controversy at the time of this writing is that Olney and colleagues have raised the question whether pediatric drugs cause immature neurons to commit suicide in the developing human brain, and critics have responded by arguing (e.g., Anand and Soriano 2004; Soriano et al 2005) that it is invalid to extrapolate data from rodent experiments to the human situation, and they have emphatically expressed the view that it would be totally inappropriate to introduce any changes in the practice of human anesthesia based on such animal experimentation. Indeed, Soriano et al 2005 argues that if such changes were introduced it “may even be deleterious to long-term neurologic outcome.”

Other critics (e.g., McClaine et al 2005) have claimed that the findings of Olney and coworkers, that anesthetic drugs kill neurons in the immature rat and mouse brain, cannot be reproduced in sheep, which they refer to as a higher order species. They suggest that the Olney et al findings can be explained either by species differences, or by failure of Olney et al to properly control cardiorespiratory function when anesthetizing immature rodents. The gist of this issue, which was also raised by Soriano et al 2005, is that because of the small size of immature rodents, it is impossible to intubate them and control their respirations while they are anesthetized. Therefore, critics claim that these animals have sustained brain damage, not due to the anesthetic drug, but due to lack of oxygen supply to the brain. McClaine et al performed their experiments on pregnant sheep, which are large enough to be maintained on a mechanical respirator that automatically controls respirations throughout the anesthesia period. Since they have maintained adequate control over respiratory function in their sheep experiments, similar to the control exercised in human anesthesia, they argue that their experiments are clinically relevant, whereas the Olney et al experiments are clinically irrelevant. On the basis of the McClaine et al 2005 findings, coauthored by 11 anesthesiologists and surgeons, it was concluded that “these results . . . corroborate the presumed safety of inhalational anesthetic use during pregnancy.” Thus, at the present time, the medical community is expressing the view that the Olney et al findings have no relevance beyond the realm of rodents, and even in rodents, the brain damage reported may be attributable to hypoxia/ischemia, and not to an effect of anesthetic drugs.

While the medical establishment does not currently consider the Olney et al findings to have direct relevance to human medicine, they do readily acknowledge that all anesthetic drugs used in obstetric or pediatric surgery are either NMDA antagonists (i.e., agents that block excitatory neurotransmission through NMDA glutamate receptors), or GABA-A agonists (i.e., agents that inhibit nerve signal transmission through GABA-A receptors). In addition, they do acknowledge that typically, these agents are used in combination, so that the brain of a neonate or young infant who requires surgery (for example, to repair a congenital heart defect) is likely to be exposed to two or more GABA-A agonists and NMDA antagonists, at the same time, during the surgery.

It also is well recognized that many anticonvulsant drugs that are typically used to treat pregnant women or infants who suffer from epileptic seizures, are either GABA-A agonists, or sodium channel blockers.

Furthermore, the entire field of pediatric sedatives also merits careful attention. For example, infants that require catheters, to administer nutrients and/or drugs (this very often includes premature babies, babies born to drug-addicted mothers, and babies with heart abnormalities or other medical problems) are usually sedated, to prevent them from pulling out their catheters. That type of sedation often lasts for days, weeks, or even months, and it directly involves drugs that suppress neuronal activity, usually via GABA receptors.

In addition, ethanol, which has both NMDA antagonist and GABA-A agonist activity as noted above, triggers severe neuro-apoptosis responses in immature brains (Ikonomidou et al 2000). It has long been known that ethanol, if ingested by a pregnant mother chronically, or even just in a small number of “binge drinking” episodes, has toxic effects on the brain of the fetus she is carrying; this condition is widely referred to as fetal alcohol syndrome (FAS). The recent findings by Olney and colleagues have elucidated the mechanism underlying that type of neurotoxic damage inflicted by ethanol on immature brains. The known vulnerability of the immature human brain to apoptogenic damage by ethanol, together with evidence that anesthetic and anticonvulsant drugs trigger neuroapoptosis in immature animal brains by ethanol-like mechanisms (i.e., through NMDA receptor suppression, and/or GABAA receptor activation) lends considerable credibility to the assumption and conclusion that anesthetic and anticonvulsant drugs very likely are indeed triggering unwanted, unnatural, and pathological levels of neuroapoptosis, in the immature and developing brains of human neonates and infants who require surgery, anticonvulsant drugs, or similar medical treatments.

There is also major public health significance in the observation that apoptotic neuronal death in neonates or infants plays an important role in the brain damage that occurs after acute brain injury in a neonate or infant (such as a hypoxic/ischemic crisis, head trauma, etc.). The reasons that support this conclusion include:

1. Acute injury episodes, in neonates and infants, frequently become sources of lifelong developmental disabilities; for example, it is believed that many cases of cerebral palsy, a lifelong disability, arise from just a few minutes of oxygen deprivation during birth;

2. Animal tests have documented that in both hypoxia/ischemia and head trauma, the magnitude of cell loss, due to delayed neuroapoptosis, is usually far greater than the magnitude of cell loss that arises directly from the initial excitotoxic insult; and,

3. Because the neuroapoptosis response occurs on a delayed time schedule, there will be a longer window of opportunity for therapeutic intervention. For example, if an infant is brought to an emergency room very soon after head trauma, the acute excitotoxic neurodegeneration will be occurring in this early period (it completely runs its course in the first 4 hrs after head trauma), but the apoptotic delayed response, which results from activity deprivation, does not begin to manifest until approximately 8 hours, at the earliest. Therefore, if the infant arrives in the emergency room 1 hour following head trauma, there is still a period of 7 hours for a neuroprotective drug to work before apoptotic degeneration would be expected to ensue. This provides an ample time window for the neuroprotective drug to “precondition” the synaptically deprived neurons, so that they will be bolstered against undergoing apoptotic degeneration. Thus, much of the delayed apoptotic degeneration may be preventable, and this represents the majority of neurons that would ordinarily die after such a traumatic event.

It should be noted that the greatest vulnerability of a neonate or infant, to developmental neuroapoptosis caused by surgical anesthetics or similar drugs, or following an acute head injury or insult, is effectively limited, or at least heavily focused, on the period of synaptogenesis. The time window during which the immature brain is most highly sensitive to damage caused by apoptogenic drugs coincides with the period of synaptogenesis, also known as the brain growth spurt period. This period occurs in different species at different times, relative to birth. In rodents, it occurs mainly postnatally (for example, in rats, it occurs during the first two or three weeks after birth). In humans, it begins in mid-gestation, and it continues (at varying rates) until about three years after birth (Dobbing and Sands, 1979).

As mentioned above, “synaptogenesis” refers to the period during which neurons establish functioning contacts (known as synapses, or synaptic junctions) with one another. It is through these synaptic junctions that nerve impulses are transmitted, and during the period of synaptogenesis, billions of such contacts are established. This is a period of rapid brain growth because, in order to receive incoming synaptic connections, each immature neuron expands its surface area by growing new branches, much like a young tree growing new branches.

All of the drugs that have been shown to trigger neuroapoptosis, in animals, during the synaptogenesis period appear to have one property in common: they abnormally suppress neuronal activity. It is known that nerve cells are programmed to kill themselves if, during development, their biological clock-like sensing mechanisms detect that they are failing to make synaptic contacts in a timely manner, and in the proper sequence. A drug that abnormally suppresses neuronal activity will disrupt the “synchrony” of the synaptogenesis process (i.e., suppressed neurons will become “out of phase” with other neurons that were not directly affected by the drug). This can cause large numbers of neurons to sense that they are out of synchrony, and effectively were not chosen for inclusion, in the synaptogenesis process of brain development. Within the desynchronized neurons, this type of perceived exclusion will automatically generate internal signals that activate the proteins that drive apoptosis. That process of neuroapoptosis, triggered accidentally and unintentionally by drugs that artificially suppress neuronal activity, will culminate in the deaths of neurons that otherwise would have survived and played their proper roles in the developing brain.

Biochemical Pathways that Mediate Immature Neuroapoptosis

The preceding discussion focuses on general principles. Once those general points have been established, attention must be turned to various specific proteins and signals that become involved in the mechanisms of neuroapoptosis.

It should be noted that apoptosis is a well-known phenomenon, which has been receiving an increasing amount of attention since the early 1970s, when it was realized that this form of cell death plays important roles in different tissues of the body, under both normal and pathological circumstances. In the ensuing years, much has been learned about apoptosis as it occurs in vitro, and in many different tissues and organs in vivo.

One common form of apoptosis prominently involves mitochondria, which are energy-producing organelles contained within all living cells. Very briefly, when various types of “upstream” processes occur, including processes that involve a death-promoting group of proteins (which notably includes a protein called Bax, and which also includes various other proteins designated as Bak, Bad, and Bid), the mitochondrial membranes become abnormally permeable. When this happens, mitochondria begin releasing a signaling protein called Cytochrome C, sometimes called the “cell death signal”.

The onset of membrane permeability, and the resulting release of Cytochrome C, create a major transition in the fate of a cell, and the following steps, which occur after Cytochrome C has been released, can be regarded as “downstream” steps. Those downstream steps include activation of an enzyme called caspase-3. This and various other enzymes trigger various changes in the cell, including “chromatin clumping” in the nucleus. In that process, the chromosomal DNA and its associated proteins (collectively called chromatin) become condensed and compacted, in ways that prevent subsequent replication of the DNA (which would be necessary for cell division), and that also prevent any messenger RNA strands (which enable protein formation) from being generated from the chromosomal DNA. Therefore, chromatin clumping (which can be seen under a light microscope) indicates irreversible cell death. It is accompanied by various other cellular processes, including the appearance of certain signaling molecules on the cell surface, which cause specialized immune system cells (called phagocytes or macrophages) to move in and finish the process of killing and digesting the cell, to release its building blocks, allowing them to be used to make new cells.

Apoptosis in the CNS is not well understood, compared to apoptosis in other tissues of the body, because there have not been any good and reliable in vivo models for studying the phenomenon in brain or spinal tissues. As a demonstration of this problem, the large majority of research in this field uses in vitro preparations of cells that are not even neurons. Those who have actually focused on neurons have most frequently used a specialized class of brain cells, known as “cerebellar granule cells,” but these cells have peculiar properties that make them uncharacteristic of neurons in general. For example, cerebellar granule cells cannot be grown in culture unless the culture medium is supplemented with high concentrations of potassium. Then, if potassium is withdrawn from the medium, these cells die by apoptosis. Since this is an easy way to produce apoptosis, many researchers have used cerebellar granule cell cultures to study neuro-apoptosis. However, the information gleaned from such studies may have little relevance to mechanisms by which alcohol and anesthetic drugs or various disease conditions trigger neuro-apoptosis in the in vivo brain. For example, Olney and colleagues have found that within 30 minutes after alcohol administration to infant mice, there is a marked suppression of the activation (phosphorylation) of ERK in the brain, and the data described herein indicate that the dropoff in ERK activation is strongly correlated with neuro-apoptosis. However, in cerebellar granule cell cultures, it has been reported that apoptosis induced by potassium deprivation is associated with an increase, rather than a decrease, in phosphorylation of ERK. Thus, based on cerebellar granule cell cultures that were studied in vitro, one would have predicted the opposite of what Olney and colleagues have actually found, in their in vivo studies. Since the literature contains large numbers of reports of apoptosis studied in vitro, using cerebellar granule cells, which appears to be directly contrary to the information Olney and coworkers are reporting, researchers in the field of neuro-apoptosis have remained skeptical of the information being reported by Olney and his coworkers.

It should also be noted that Olney and his coworkers did not make their discoveries by focusing on in vitro studies of apoptosis. They began studying head trauma, in the late 1990s, and the data they gathered clearly indicated that head trauma causes neurons to die by two completely different mechanisms, which are excitotoxic cell death, and apoptotic cell death. Since those two damage processes involve very different mechanisms, they began trying to sort out and separately identify and evaluate the roles and contributions of each of those two mechanisms, in the overall damage process. As part of that process, they began screening various drugs, to determine whether any such drugs could prevent either or both of those different types of neuronal damage.

The results of those tests showed that certain drugs which could help reduce excitotoxic neuronal death, actually made the apoptotic neuronal damage much worse. Subsequently, when those same types of anesthetic or similar drugs were administered to normal infant animals that had not been subjected to head trauma, they triggered a robust neuro-apoptosis response, in the normal brain. Those results are described in articles such as Jevtovic-Todorovic et al 2003, and Olney et al 2004a and 2004b.

Those results, showing serious brain damage in normal uninjured infant animals that were given anesthetic or similar drugs, was totally unexpected and very surprising, because many of the drugs that clearly triggered robust neuro-apoptosis, in infant animals, are anesthetic, sedative, and anti-convulsant drugs that are widely and actively used today, in pediatric and obstetric medicine on humans.

Understandably, clinicians who continue to treat babies and infants with those types of anesthetic, sedative, or anti-convulsant drugs have regarded the findings of the inventor herein with distrust and disbelief, and the inventor's findings and reports have been directly and aggressively disputed and criticized, by numerous experts in the field. For example, as mentioned above, in publications such as Anand and Soriano 2004, Soriano et al 2005, and McClaine et al 2005 (which was coauthored by 11 anesthesiologists and surgeons), numerous experts have directly, openly, and publicly challenged the findings of the inventor herein. There is no indication in the literature that physicians, researchers, or authoritative medical organizations are recognizing that neuro-apoptosis, possibly induced by surgical anesthetics and certain other drugs that continue to be widely used in pediatric and obstetric medicine, may be posing serious problems, dangers, and risks, to fetuses, neonates, and infants. If the findings and reports of the inventor herein were believed and accepted by experts, those findings would compel and obligate those physicians and anesthesiologists to actively adopt and begin using corrective measures, to prevent such drugs from causing lifelong brain damage in the human patients they continue to treat with such drugs. However, rather than responding in that manner, experts in this field have responded by attacking and criticizing the findings of the inventor herein.

It should also be noted that, once Olney and his coworkers discovered and demonstrated that neuro-apoptosis could be clearly identified and evaluated, in in vivo brains, by methods that distinguish apoptotic damage from excitotoxic damage (as described in Ishimaru et al 1999, Olney 2003, Young et al 2004, and Bayly et al 2006), they used those same testing methods to study the brain damage that occurs in “fetal alcohol syndrome”, in the brains of babies born to mothers who are addicted to alcohol, or who engage in one or more sessions of “binge drinking” during a pregnancy. The results of that research, described in Ikonomidou et al 2000 and Olney et al 2002a, made it clear that: (i) ethanol will trigger a robust neuro-apoptotic response, in immature brains; and, (ii) the neuro-apoptosis is caused by chemically-induced neuronal inactivity, which activates the “pruning” mechanism described above, which will kill brain neurons during synaptogenesis, if it appears (from apparent inactivity) that the neurons have not become integrated into the active neural networks of the developing brain.

Accordingly, those recent discoveries provided new research models and approaches for in vivo study of neuronal apoptosis in immature brains. Using those methods and models, Olney and colleagues determined (Dikranian et al 2001, Olney et al 2002b, Young et al 2003 and 2005) that alcohol-induced or anesthetic-induced neuro-apoptosis conforms to the mitochondrial model, as described above. It is a Bax-dependent process, involving: (i) translocation of activated Bax proteins to mitochondrial membranes; and, (ii) disruption of membrane integrity by Bax proteins, which leading to membrane permeability, and the release of Cytochrome C. The released Cytochrome C triggers the “downstream” steps referred to above, culminating in activation of caspase-3 enzymes, leading to terminal degradation and death of the affected cells. Thus, in alcohol-induced or anesthetic-induced neuro-apoptosis in in vivo immature brains, mitochondria have been shown to play a key role in defining the transition from a living cell, to a cell that is irreversibly committed to a rapid death. After Bax protein alters the integrity of the mitochondrial membrane, releasing Cytochrome C, a death cascade begins, and even if it is somehow interrupted or partially blocked or delayed, the cell is almost certainly doomed. These biochemical steps in the CNS apoptosis cell death process are presented schematically in FIG. 1.

Accordingly, one object of this invention is to disclose what is currently known about the risks that certain classes of drugs (including anesthetics used in obstetric or pediatric surgery) pose, in terms of inflicting permanent brain damage on fetuses, neonates, and infants, by mechanisms that involve: (i) disruption of the essential process of synaptogenesis, in immature brains; and, (ii) triggering of pathological forms of neuro-apoptosis, caused by the disruption of synaptogenesis within immature brains.

Another object of this invention and application is to disclose combinations of agents that can be used to provide surgical anesthesia or other necessary medical treatments for fetuses, neonates, or infants, without substantially disrupting the essential processes of synaptogenesis within immature brains, and without creating unacceptably high risks of inflicting permanent brain damage on the treated patients.

Another object of this invention and application is to disclose testing and evaluative criteria that can be used to identify other additional agents that can provide effective medical treatments for fetuses, neonates, or infants, without disrupting the critical processes of synaptogenesis in immature brains, and without creating unacceptably high risks of inflicting permanent brain damage on such patients.

These and other objects of the invention will become more apparent through the following summary, description, and examples.

### SUMMARY OF THE INVENTION

Drug treatments are disclosed for preventing pathological brain damage that can be caused by surgical anesthetics and certain other compounds or crises, in fetuses, neonates, and infants. In the developing brains of immature mammals, a surplus of neurons is initially created. Neurons that do not become actively integrated into functioning neural networks are killed and deleted, by a process of “programmed cell death” called apoptosis, which is controlled by mitochondria. During the development stage called synaptogenesis, immature neurons are very sensitive to activity deprivation; a sequence of events will initiate apoptotic death in such neurons, if they are deprived for even a short period of time (such as a few hours) of signals or activity that otherwise would indicate active engagement and involvement in the developing brain. This type of “activity deprivation”, which can trigger pathological neuronal death leading to permanent brain damage and mental impairment, can result from hypoxic/ischemic crises, or from head injury or other trauma; it also can be caused by exposure to ethanol, or to various sedative, anti-convulsant, or anesthetic drugs, including the surgical anesthetics that are commonly used on neonates or infants.

That type of pathological neuroapoptosis, and the permanent brain damage it causes, is largely preventable, using methods and compositions described herein, such as by administration of a lithium salt to a pregnant mother, neonate, or infant, prior to surgery, or within a few hours after a traumatic head injury or a hypoxic and/or ischemic crisis. With regard to neuroapoptotic cell death following a head trauma or hypoxic/ischemic crisis, apoptotic destruction does not begin killing immature neurons until about 8 hours after the traumatic event; this provides physicians with sufficient time to administer preventive drugs (such as lithium) that will intervene in the process, by removing or altering one of the links in the chain of apoptotic events. With regard to surgical anesthesia involving fetuses, neonates, or infants, improved anesthetic regimens are disclosed that will avoid or minimize the apoptotic destruction cascade. In addition, these discoveries can also be used to minimize brain damage in fetuses carried by alcoholic and/or binge-drinking mothers.

The invention arises from several observations and insights, which include the following:

1. Survival of developing neurons is regulated by several intracellular signaling pathways, all of which have a common denominator. They all regulate cell survival by influencing the homeostatic balance between death-promoting (e.g., Bax and Bak) and survival-promoting (e.g., Bcl-2 and Bcl-xL) members of a Bcl-2 multidomain family of proteins. Under normal circumstances, the balance is maintained in favor of the survival-promoting factors, and the death-promoting factors are held in check. However, activity deprivation in immature neurons (caused by anesthesia, alcohol, hypoxia or ischemia, etc.) can alter certain intracellular signaling pathways, in ways that will shift the balance. This enables death-promoting factors (including Bax and Bak proteins) to become dominant, causing pathological neuronal apoptosis in fetuses and infants, leading to permanent brain damage.

2. One mechanism by which activity deprivation can trigger neuroapoptosis, in immature neurons, involves suppression of the ERK MAP kinase signaling pathway. Therefore, treatments that increase ERK MAP kinase activity can prevent or reduce the types of pathological neuroapoptosis and brain damage that are triggered by activity deprivation in immature neurons.

3. Agents that can increase ERK MAP kinase activity include lithium (a potent protective agent, which is well-known in adult medicine, but which has not previously been used in neonatal or pediatric medicine), and xenon (which has its own anesthetic properties). Other candidate agents can be evaluated for their ability to increase ERK MAP kinase activity, by means of in vitro (cell culture) tests as well as in vivo animal tests.

4. Another mechanism by which activity deprivation triggers neuroapoptosis in immature brains is by suppressing the PI3K/AKT signaling pathway. Pharmacological agents that can penetrate blood-brain barriers and activate the PI3K/AKT pathway are known, including antagonists of beta-1 adrenergic receptors, and agonists of beta-2 adrenergic receptors. Although these agents are not as potent as lithium in protecting against immature neuroapoptosis, such agents nevertheless can be used, and can provide protective benefits in at least some cases.

5. PKA and PKC signaling pathways also have regulatory influences on the Bcl-2 family of death/survival proteins. Accordingly, pharmacological agents that act on the PKA and/or PKC signaling pathways are promising candidates for preventing immature neuroapoptosis. As examples, drugs that activate muscarinic cholinergic receptors are functionally linked to PKA and PKC signaling pathways, and also increase activity in the ERK MAP kinase and the PI3K/AKT pathways. Accordingly, muscarinic cholinergic agonists can help protect neurons, in at least some regions of the brain, against neural apoptosis caused by pediatric/obstetric drugs that suppress neuronal activity.

### BRIEF DESCRIPTION OF THE DRAWINGS

FIG. 1 is a schematic depiction of an immature neuron during the developmental stage known as “synaptogenesis”, indicating several major steps that will occur if apoptosis is triggered by a period of “activity deprivation”. While passing through that vulnerable stage, if neuronal activity is artificially inhibited by ethanol, a surgical anesthetic, or a similar drug, a series of “upstream” events will occur, culminating in activation of Bax signaling proteins, which will indicate to the mitochondria in the cell that the neuron is a surplus neuron that has not been connected into the neuronal networks of the brain, and it should be destroyed and deleted. The Bax signals will cause the mitochondrial membrane to become permeable, allowing the release of a signaling protein called Cytochrome C, also called the “cell death signal”. The release of Cytochrome C triggers a series of “downstream” events involving Apaf-1, caspase-9, and caspase-3 proteins, which drive the rapid death and destruction of the neuron, so that its building blocks can be recycled and used to form other nonneuronal cells in the growing fetus or infant.

FIG. 2 depicts a number of signaling pathways that participate in determining whether a neuron in a fetal or infant brain will live, or be destroyed and deleted by apoptosis. These signaling pathways influence the homeostatic balance between the “Bcl” family of proteins (which hold apoptosis in check, allowing a neuron to live and become part of the brain), and the Bax and Bak family of proteins (which tip the balance in favor of apoptosis and cell destruction, if an absence of neuronal activity indicates that some particular neuron is a surplus neuron that did not become connected in the neuronal networks being formed in the brain). If one or more of these signaling pathways are artificially suppressed (such as by ethanol, a surgical anesthetic, or a similar drug, or by a head injury or hypoxic/ischemic crisis in some portion of the brain), the balance will shift in favor of the pro-death Bax and Bak signaling proteins. An intervention that activates one or more of these pathways can reduce or prevent pathological neuroapoptosis from killing neurons that should have been spared and allowed to live.

FIG. 3 (which comprises graphs 3A, 3B, and 3C) shows how ethanol administration in immature mice will suppress activity in three of the signaling pathways illustrated in FIG. 2.

FIG. 4 illustrates the apoptotic response that is induced in the caudate nucleus region of the infant mouse brain by isoflurane or midazolam (anesthetics that act at GABA receptors). FIG. 4 also indicates that pilocarpine (which activates muscarinic cholinergic receptors) markedly reduces neuronal damage in infant mice caused by either of those anesthetics.

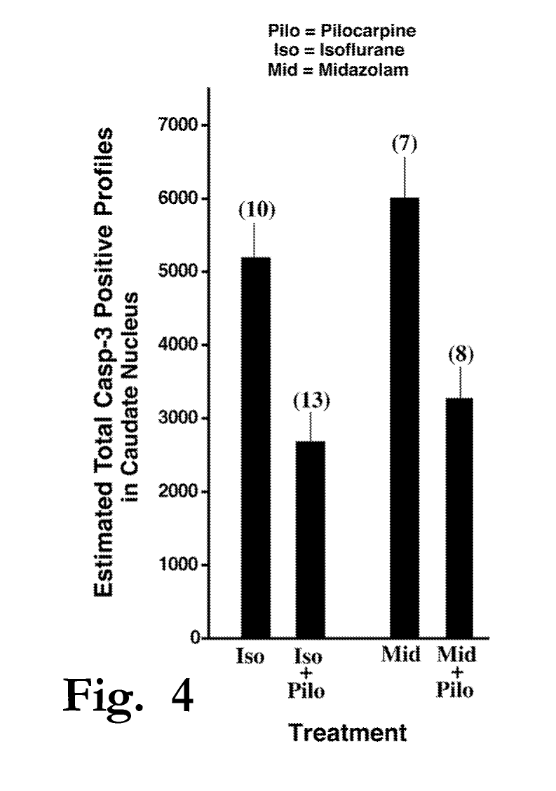

FIG. 5 shows that ethanol (i.e., ethyl alcohol, the toxicant that causes fetal alcohol syndrome) induces a strong neuroapoptotic response in infant mice. It also shows that lithium potently suppresses and can completely prevent that type of neuronal damage.

FIG. 6 shows that the surgical anesthetics ketamine (which suppresses NMDA receptors) and propofol (a GABA agonist) can each induce severe and pathological neuroapoptosis in infant mice. It also shows that lithium can potently prevent the type of damage caused by surgical anesthetics in immature brains, regardless of whether the anesthetics act at NMDA receptors, or at GABA receptors.

FIG. 7 shows that the inhalational anesthetic drug, xenon, triggers a mild neuroapoptotic response in an infant mouse brain. It also shows that isoflurane, another inhalational anesthetic, causes a much more severe neuroapoptosis response. However, when both anesthetics are administered together, xenon markedly protects against the isoflurane-induced neuroapoptosis.

FIG. 8 illustrates the protective effects of lithium (Li) when administered to infant mice, 4 hours after a traumatic brain injury (TBI) was inflicted on one side of the head. The bars depict the amount of neuroapoptosis (indicated by activated caspase-3 levels) in two separate regions of brain tissue, after TBI alone (in control animals), or after TBI combined with lithium treatment. In the vertical scale, the 100% level (indicated by a dashed horizontal line) indicates the normal “baseline” levels of caspase-3, in brain tissue from the contralateral (uninjured) side of the brain. As shown by these data, lithium greatly reduced and prevented apoptotic neuronal damage caused by traumatic brain injury in immature animals, even though the lithium was not administered to the animals until four hours after the injury.

### DETAILED DESCRIPTION

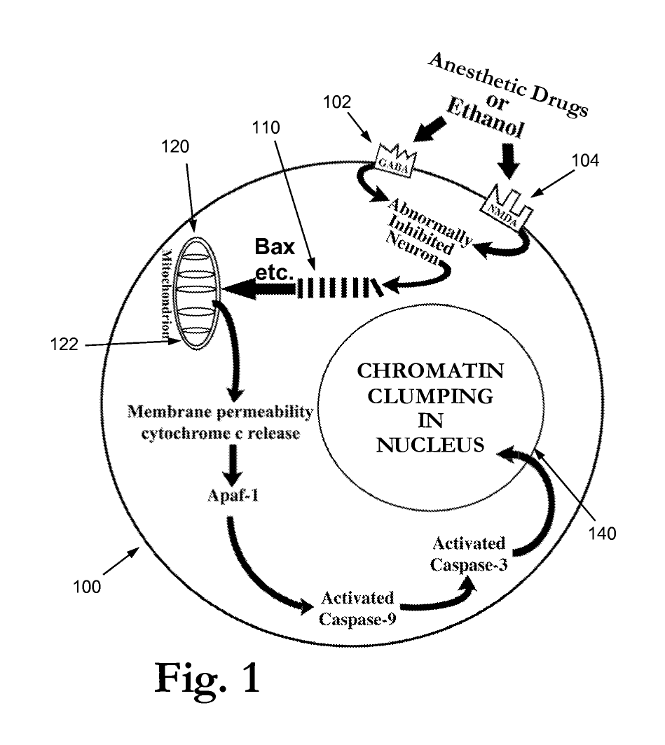

As summarized above, and as depicted schematically in FIG. 1, pathological forms of neuro-apoptosis in an immature brain (i.e., during fetal, neonatal, or infant development, such as up to about 3 or 4 years of age in humans) can be caused by even brief periods (such as a few hours) of activity deprivation, if those periods of activity deprivation occur during critical and vulnerable stages of synaptogenesis, when the brain must determine which neurons to keep, and which ones to delete. Such activity deprivation can be caused by ethanol intake by a pregnant female, or by various types of anti-convulsant, sedative, or anesthetic drugs (such as surgical anesthetics, sedating drugs used to keep babies from pulling out catheters, etc.).

FIG. 1 schematically depicts an immature neuron 100 that is being subjected to surgical anesthesia, or to a “binge drinking” episode by an alcoholic mother. Either agent will artificially suppress neuronal activity, by acting at GABA receptors 102 and/or NMDA receptors 104, located on the outer membrane surface of immature neuron 100.

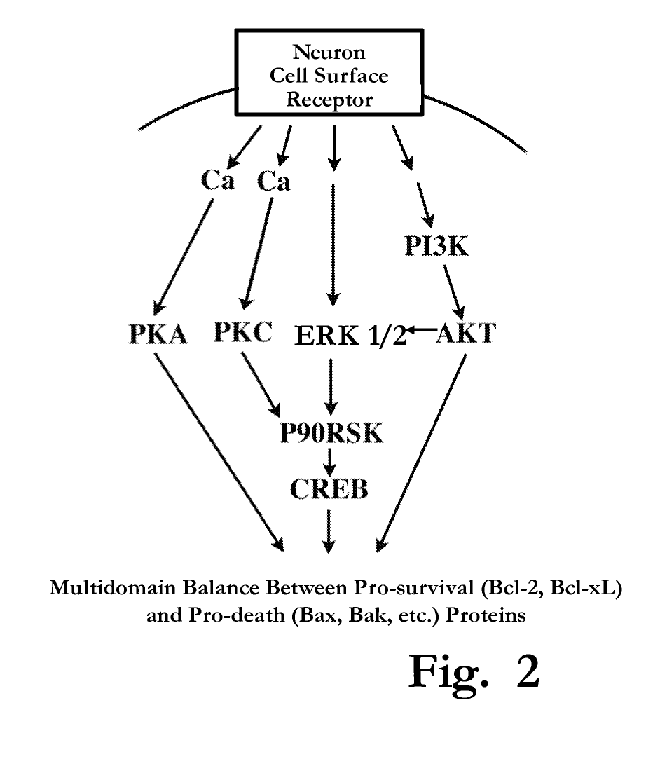

Inside neuron 100, a series of events that involves several distinct yet interrelated signaling pathways, represented by a row of dominoes 110 (several of these signaling pathways are identified below, and in FIG. 2), will lead to activation of a set of “pro-death” proteins (also called “pro-apoptotic” proteins). As described in the Background section, these include several proteins designated as Bax, Bak, Bad, and Bid; for convenience, they are designated in FIG. 1 as “Bax etc.” proteins. Normally, these proteins float in the liquid (usually called the cytosol or cytoplasm) that fills most of a cell. When they are activated, at least some of these proteins (including the Bax protein) translocate to the external surfaces of the mitochondria in a cell. Each cell holds dozens or hundreds of mitochondria, which are small organelles that are enclosed within their own membranes. The mitochondria are schematically represented in FIG. 1 by a single mitochondrion 120, enclosed within mitochondrial membrane 122.

When a mitochondrial membrane 122 is contacted by activated Bax proteins (and possibly other activated pro-death proteins), the membrane 122 becomes permeable. This allows the release of a signaling protein called Cytochrome C. As mentioned above, Cytochrome C is sometimes called the “cell death signal”.

The transition to mitochondrial membrane permeability, which is indicated by the release of Cytochrome C, is regarded as a boundary or transitional stage. It divides and separates the overall sequence of steps that lead to the apoptotic death of a neuron into a first set of “upstream” steps (which lead to mitochondrial membrane permeability and the release of Cytochrome C), and a second set of “downstream” steps (which begin to occur after the mitochondrial membranes become permeable and Cytochrome C is released). That distinction is crucial, because effective drug interventions, to prevent the types of pathological neuro-apoptosis that can be caused in immature brains by drug- or alcohol-induced activity deprivation, must prevent or suppress one or more of the “upstream” steps. In other words, to be effective, the treatments disclosed herein must act by means of mechanisms that can prevent or suppress mitochondrial membrane permeability, and prevent the release of Cytochrome C by mitochondria inside the neuron. In FIG. 1, that series of upstream steps, leading to activation of Bax etc. proteins, is represented by the row of dominoes 110.

That set of “upstream” signaling steps is depicted in more detail in FIG. 2, which identifies a number of signaling pathways that offer candidates for potentially effective intervention to prevent brain damage, using the methods and agents described herein. In general, any signaling pathway that leads to: (i) activation of the Bax, Bak, Bad, and/or Bid signaling proteins, and/or (ii) translocation of any of those activated proteins to mitochondrial membranes, are part of the “upstream” sequence of events in apoptosis, and therefore offer good candidates for intervention, to prevent the types of pathological neuroapoptosis that can be caused in immature brains by drug- or alcohol-induced activity deprivation.

By contrast, once the mitochondrial membrane 122 has become permeable (as evidenced by the release of Cytochrome C by the mitochondria), the “downstream” stages of apoptosis will commence. As indicated in FIG. 1, this includes activation of Apaf-1, caspase-9, and caspase-3 proteins, which will then shut down and inactivate the nucleus 140 of a cell. The changes that will be triggered, in a cell nucleus, include the “clumping” of DNA and its associated proteins (collectively referred to as “chromatin”) into compacted, condensed globular structures. That type of clumping effect will block and terminate the ability of the cell's DNA to create mRNA strands that encode proteins, which normally would be created in an ongoing manner by a healthy cell.

It is not conceded herein that it is impossible to intervene and stop one or more of those “downstream” protein activations or activities, in ways that can rescue immature neurons; indeed, such efforts merit evaluation, in view of their potential to help rescue immature neurons and minimize permanent brain damage in immature brains. However, because of practical factors, it should be recognized that earlier interventions, designed to prevent the “upstream” steps that lead to activation of Bax etc. proteins and the release of Cytochrome C (the “cell death signal”), offer greater promise for providing useful and efficient interventions.

In particular, it should be recognized that dozens or hundreds of mitochondria, in a single neuron, may be releasing substantial numbers of Cytochrome C molecules in that neuron, and any of those Cytochrome C molecules may be able to relaunch and restart the downstream death cascade once again, even if an initial set of activated Apaf-1, caspase-9, or caspase-3 molecules were blocked and inactivated by a pharmaceutical treatment. Furthermore, even if methods or agents can be identified for blocking some particular step in a “downstream” pathway, such efforts might lead to undesired outcomes, if they end up rescuing (or prolonging the deaths of) neurons that are severely injured. Once a neuron reaches a stage of mitochondrial membrane permeability leading to Cytochrome C release, it may become, in effect, a badly injured and potentially crippled neuron, which may end up inflicting disruptive and adverse effects on the neural networks that are being created in a developing brain.

Nevertheless, these types of “downstream” interventions are worth studying in animal models, since they might lead to useful therapeutic benefits (as evidenced by behavioral, memory, or similar tests on the animals), and since the data gathered in such research may end up shedding additional light on various processes of neuroapoptosis, which (as noted above) are poorly understood under the prior art.

As described in greater detail below, and as illustrated in FIG. 2, the Applicant herein has identified certain proteins (AKT, ERK-1, and ERK-2) in the upstream apoptosis pathways that are markedly altered, in brain tissue from immature animals, within 30 minutes after ethanol administration. In this early period, expression of the active (phosphorylated) isoforms of these proteins are markedly suppressed. Both of these proteins have been identified as proteins that regulate cell survival, by means that appear to require a steady and gradual low-level supply of the phosphorylated isoform. If the gradual production of these active isoforms is hindered or blocked, their absence will contribute to switching the balanced signaling mechanisms from a pro-survival balance (which is manifested by sustained levels of certain proteins, including Bcl-2 and Bcl-xL, as indicated at the bottom of FIG. 2), toward a pro-death, apoptosis-triggering status (which is manifested by increased levels of activated Bax and Bak proteins). Thus, the AKT, ERK-1, and ERK-2 proteins (and various other related proteins belong within the “upstream” steps of apoptosis, as represented by dominoes 110 in FIG. 1) offer good targets for pharmaceutical intervention to prevent pathological neuroapoptosis, using agents such as described below.

A number of candidate agents for use in such interventions are already known and available, as listed and described below, and other such agents can be identified, by means of relatively simple screening processes, now that the pathways and mechanisms leading to drug-induced or alcohol-induced pathological neuroapoptosis have been identified and explained. For example, a candidate treatment drug can be administered to an infant mouse or rat, at one or more dosages that are believed to fall within or cover a suitable dosage range. At the same time, or shortly before or after such treatment, ethanol or a suitable anesthetic or anticonvulsant drug (several such drugs are identified in the Examples) is also administered, at a dosage that, when administered alone, will reliably cause a substantial neuroapoptotic response in animals of that type and age. Four to eight hours later, the animal is sacrificed, and its brain is examined (using methods such as described in the Examples) to see if the neuroapoptosis response was reduced, or prevented, by the candidate drug.

As described above, head trauma, hypoxia/ischemia or other crises, or treatments using sedatives, anesthetics, or anticonvulsants that suppress neuronal activity, can place immature neurons in activity-deprived conditions that can trigger apoptosis. Thus, effective prophylaxis will require an approach that prevents the deprived condition from triggering a suicide signal in some or all of the affected neurons. The subject invention utilizes two separate approaches, either of which can prevent the suicide signal from being generated or acted upon in some or all of the neurons that would die if not protected. These approaches are: (1) activation of whole neurons, and (2) activation of signaling pathways within neurons.

### Activation of Whole Neurons

Since neuronal suppression (deactivation) is the condition that can cause immature neurons to commit suicide, one approach for preventing unwanted and pathological neuronal death involves counteracting a deactivation condition with a stimulus that activates neurons. A potential problem with this approach is that an anti-convulsant or anesthetic drug, in order to provide effective therapy, must achieve a substantial degree of neuronal suppression. However, an important consideration is that anti-convulsant and anesthetic drugs perform their actions in excess. For example, the most important action of NMDA antagonists for surgical anesthesia purposes is their analgesic action, which is largely achieved by blocking NMDA receptors at certain levels of the spinal cord where NMDA receptors receive pain messages from various parts of the body and relay them up to the brain. There are NMDA receptors on neurons distributed widely throughout many other brain regions, pertaining to neural circuits that mediate other functions that are irrelevant to analgesia or anesthesia. If the deactivating action of anesthetic drugs on neurons throughout these other neural circuits can be counteracted by an activating stimulus that does not interfere with the blockade of NMDA receptors in the spinal pain pathways, many neurons can be saved from becoming suicide victims, while the purposes of anesthesia are also being served.

The same is true for GABA drugs. In addition to their actions in circuits relevant to anesthesia, they also have actions in many other circuits that are irrelevant to anesthesia.

Therefore, one approach disclosed herein involves including, in an anesthetic or anti-convulsant protocol that is to be applied to a neonate or infant, a pharmacological agent that is capable of selectively counteracting the suppressant action of NMDA or GABA drugs, in neuronal circuits that are irrelevant to anesthesia, or to anti-epileptic or other anti-seizure therapy.

In certain brain regions, such as the caudate nucleus, both GABA and NMDA anesthetic drugs trigger a dense pattern of neuroapoptosis, because neurons in this brain region are well-endowed with both GABA-A and NMDA receptors. However, the suppressant actions of such drugs, within the caudate nucleus portion of the brain, are entirely irrelevant to their desired anesthetic or anti-convulsant actions, which are mediated through other circuits.

Caudate neurons are also well-endowed with the muscarinic subclass of cholinergic receptors, which can be activated by various known cholinergic agonist drugs, such as pilocarpine. The Applicant has observed that if pilocarpine is administered to infant mice, prior to administration of a GABA anesthetic drug at a dosage that otherwise would cause serious or severe neuroapoptosis, the degeneration of caudate neurons can be completely prevented, or greatly reduced. Therefore, administering a cholinergic agonist (such as pilocarpine) as a pretreatment, prior to administering a GABA anesthetic agent (such as midazolam or isoflurane), offers a method for carrying out the invention disclosed herein. Extending that method to a composition of matter and a packaged product, this invention discloses a composition of matter, and an article of manufacture, containing an appropriate amount of a cholinergic agonist drug for intravenous administration to a neonate or infant, in timed sequence with an appropriate amount of a GABA drug that can induce or maintain anesthesia in a neonate or infant.

While cholinergic activation is discussed herein as a method of activating whole neurons, it is possible that its effectiveness in preventing neuroapoptosis may depend on a more complicated mechanism involving activation of one or more intracellular signaling pathways, as discussed in the next section. Alternately or additionally, the efficacy of preventing neuroapoptosis by activating whole neurons, using one or more specific types of drugs such as muscarinic cholinergic agonists, might be increased and enhanced by also using one or more additional drugs, such as described below, to also target and modulate certain specific intracellular signaling pathways.

### Modification of Intracellular Signaling Pathways

Pathological apoptosis of immature neurons, when triggered by activity deprivation, involves alterations in the activity states of specific molecular entities that mediate intracellular functions relevant to neuronal division, differentiation, migration, and/or synapse formation. How well these molecular entities perform their functions is a major determinant of whether the neuron will survive or perish (by apoptosis) during the course of development. Thus, these molecular pathways are, in effect, regulators of cell survival. The molecular entities that mediate these processes are primarily proteins, and they are organized in systems that are sometimes referred to as cascades, in which activation of a first molecule in the system causes activation of a second, which then activates a third, and so forth.

These proteins usually are identified by acronyms that arise from descriptive phrases. As examples, MAP kinases (often abbreviated as MAPK's) are mitogen-activated protein kinases; CREB proteins are cyclic AMP (adenosine monophosphate) response element binding proteins; and ERK proteins are extracellular signal regulated kinase proteins. These acronyms, and the proteins to which they refer, are well-known among those skilled in the art, and are described in numerous articles that discuss various aspects of apoptosis, cancerous cell growth, and other cellular and physiological processes. Review articles that discuss these proteins (citing other articles that describe these proteins in more detail) include Strasser 2000, Cheung 2004, Hsieh 2005, Rubinfeld 2005, Hetman et al 2006, Dell'Acqua 2006, Zhuang et al 2006. Since the claimed priority date of this invention is Aug. 2, 2004 (the filing date of provisional application 60/598,390, which led to PCT application PCT/US2005/027460 in August 2005), the listing of articles published on or after August 2004 does not concede that such items are prior art against this invention. Nevertheless, any articles can be consulted, regardless of date, for information about the molecular pathways involved in the “upstream” stages of apoptosis, which lead to mitochondrial membrane permeability, and to the release of Cytochrome C by mitochondria, as indicated schematically in FIG. 2.

As briefly mentioned in the Background section, some recent articles, including Cheung et al 2004 and Zhuang et al 2006 as examples, teach directly away from this invention. Those articles (many of which are based on in vitro tests using cerebellar granule cells, which are an atypical class of cells, as mentioned in the Background section) teach that activation of ERK proteins will promote apoptotic neuronal death. In direct contradiction to those teachings, data gathered by the inventor herein (as described below) strongly indicate that agents which activate ERK-1 and ERK-2 proteins can dramatically suppress and prevent the pathological neuro-apoptosis that is caused when ethanol or anesthetic drugs are administered to immature animals, or that is caused by traumatic head injury.

On the subject of ERK proteins, it should be noted that these “extracellular signal-regulated kinase” proteins are intracellular proteins, and their name indicates that their activation is regulated by extracellular signals. Typically, such signals involve an extracellular molecule (such as a neurotransmitter, hormone, or cytokine) binding to and activating a receptor having an exposed and accessible binding domain on the outer surface of a neuron. That type of extracellular reaction will lead to phosphorylation or other activation of ERK and other signaling proteins located inside the receptor-bearing neuron.

### The ERK-MAP Kinase System as a Target for Modification

Unpublished evidence generated by Olney and colleagues indicates that a MAP kinase cascade plays a central role in mediating neuro-apoptosis induced by deprivation circumstances. MAP kinases were originally studied as regulators of mitosis and more recently have been found to be abundantly expressed in the cell bodies and dendrites of post-mitotic neurons, where it is believed they regulate differentiation, synaptogenesis, and survival of neurons.

Three MAP kinase pathways have been identified. These are distinct and non-interactive pathways, each of which has a three-tiered system in which activation of a first kinase (MKKK) leads to activation of a second kinase (MKK), which activates a third kinase (MAPK).

In one system that is of interest in this invention, the third kinase in that type of cascade is referred to as an ERK (i.e., an extracellular signal-related kinase protein, as indicated above). The ERK-MAPK cascade is of special interest in relation to deprivation-induced neuro-apoptosis, because it is known that activation of the NMDA receptor causes an increase in the activated (phosphorylated) isoform of ERK, which triggers translocation of ERK to the nucleus, where it indirectly causes phosphorylation of CREB, a transcription factor that can directly or indirectly activate various genes that control protein synthesis.

Some of the proteins that are synthesized via this pathway are members of the multidomain “Bcl” family of proteins, which regulate cell survival (“Bcl” refers to B-cell lymphomas, which were the cell types in which Bcl proteins were first identified). Proteins such as Bcl-xL and Bcl-2 are anti-apoptotic, i.e., they act to prevent apoptosis from occurring, thereby sustaining and protecting a cell. Other proteins, including proteins in the “Bax” and “Bak” families, are pro-apoptotic.

Normally, the pro-apoptotic (death promoting) and anti-apoptotic (survival promoting) proteins are maintained in homeostatic balance, by CREB-regulated synthesis of each protein at a rate conducive to an anti-apoptotic balance, which supports cell survival. However, if that balance is altered and tipped in a pro-apoptosis direction, by a force external to the system (e.g., by the influence of ethanol, anesthetic drugs, or loss of synaptic inputs), the pro-apoptosis components assume control, and an apoptosis signal is generated and acted upon by Bax, a major effector protein. When activated, Bax proteins will translocate to mitochondrial membranes, and will initiate a cascade of events leading to mitochondrial membrane permeability. This allows the release of Cytochrome C, the signaling molecule that will initiate the “downstream” steps of apoptosis, which will culminate in apoptotic death of the neuron.

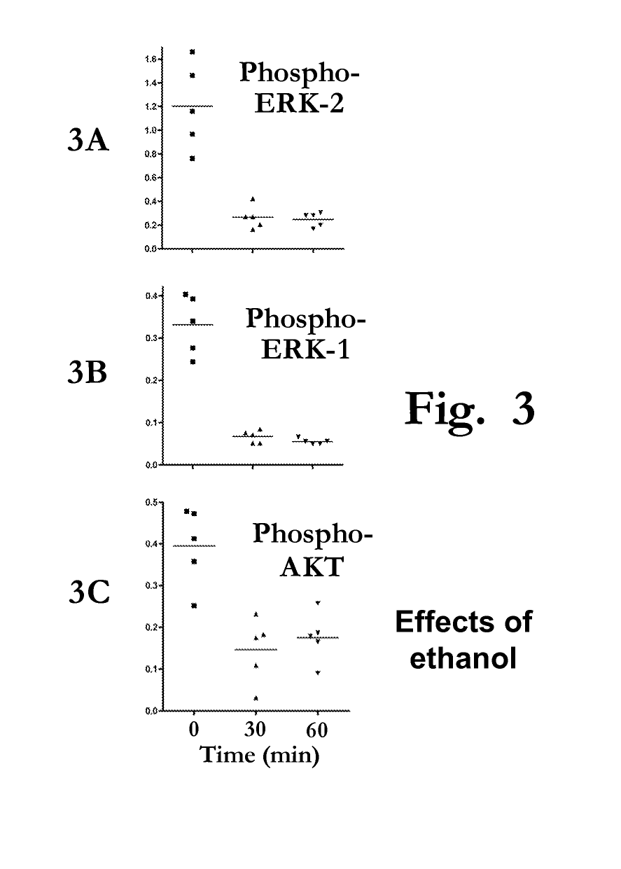

The graphs in FIGS. 3A and 3B indicate that administration of ethanol (a potent suppressor of neuronal activity, which has both NMDA antagonist and GABA agonist activity) to infant mice will cause a rapid decrease (within less than 30 minutes) in the activated isoforms of ERK-1 and ERK-2. This signifies that the NMDA-receptor-linked ERK/CREB pathway is suppressed by ethanol, in ways that can disrupt the balance of protein factors that regulate cell survival, altering the balance in ways that promote neuro-apoptotic cell death. In this case, decreased neuronal activity, believed to be acting mainly through NMDA receptors, contributes to the reduced levels of activity in the ERK/CREB pathway.

In the case of a head trauma (or an ischemic/hypoxic crisis or other assault on the head or brain), a loss of synaptic inputs, from the neurons that were injured by the trauma or other crisis, would be the initiating step that would cause the ERK/CREB pathway to be suppressed. The loss of arriving signals, which will drop off if the signal-generating neurons are severely damaged, would have the effect of silencing excitatory activity that would be mediated by the same NMDA receptors that can be driven into silence by ethanol, anesthetic drugs, etc.

Although the ERK-CREB pathway is not the only mechanism by which deprivation circumstances can trigger neuroapoptosis, it nevertheless should be recognized that deactivation of the NMDA receptor-linked ERK-CREB pathway can disrupt the homeostatic balance between pro- and anti-apoptosis proteins, thereby allowing pro-apoptosis proteins (culminating in Bax activation and translocation) to gain ascendancy, trigger apoptosis.

The present invention discloses methods for interceding in a manner that can stabilize the ERK-CREB pathway, thereby restoring the homeostatic balance between pro- and anti-apoptosis proteins in favor of neuronal protection and survival. There are various known intermediary factors in this pathway, any of which offer appropriate targets for intervention, and additional factors may be discovered in the future that will offer still more targets for protective intervention. It is important to note that these targets are part of the “upstream” stages of the apoptosis cascade, which offers important advantages, as mentioned above.

For example, if suppression of ERK-1 and/or ERK-2 activation (which involves phosphorylation of those ERK proteins) is counteracted by a pharmaceutical intervention that promotes ERK phosphorylation, this can help abort and prevent the process of pathological neuro-apoptotic signaling, very nearly at its inception. In this type of treatment, suppression of NMDA receptor activity by an anesthetic drug can be carried out, to enable surgery; however, just as the ERK system is being negatively impacted, a counteracting positive force can stabilize the system, in ways that will prevent a pathological signaling cascade from propagating through the complete sequence of an “upstream” apoptosis-triggering sequence that otherwise would lead to Bax activation, Cytochrome C release, and cell death.

The lithium ion offers one example of a candidate agent for achieving this purpose. It is known that lithium influences a number of intracellular signaling processes in ways that can affect various types of neurological processes; for example, one well-known use is for treating bipolar disorder, often called manic-depressive illness. For lithium to have its beneficial effects, it requires hours or preferably days of administration, during which time its influence in stabilizing intracellular signaling pathways will build up to stable levels. One such stabilizing influence that is documented in the literature is that lithium stimulates increased activation of the ERK MAP-kinase system (Einat et al 2003), in ways that can be used, via CREB activation as mentioned above, to increased synthesis of the anti-apoptosis (pro-survival) protein Bcl-2. Therefore, lithium could be administered for a few days prior to surgery, on a pediatric patient who must undergo prolonged anesthesia (such as to correct a heart defect). The lithium buildup, during the presurgical period, would shift the ERK-CREB pathway to a level higher than normal. This would help prevent a short-term decrease in that pathway (caused by surgical anesthesia) from initiating a signal cascade that would lead to pathological neuro-apoptosis.

Xenon, an inert gas, is also a good candidate drug for achieving the purposes of this invention. Xenon is known to have powerful analgesic and anesthetic properties, but it has not been commercialized for that purpose, primarily because it is rare, and costly to extract from the atmosphere in large quantities. It was demonstrated by Franks et al 1998 that xenon has NMDA antagonist properties, which can explain its analgesic and anesthetic actions. While its NMDA antagonist properties would theoretically cause xenon to trigger neuroapoptosis in an immature brain, xenon has an important offsetting property. It stimulates a neurotrophic factor pathway involving brain-derived neurotrophic factor (BDNF). The BDNF pathway, independent of NMDA receptors, feeds into and regulates the ERK-CREB pathway. Xenon has been shown (Ma et al 2005) to up-regulate the BDNF-ERK-CREB pathway, in ways that can result in increased synthesis of the cell survival-promoting protein, Bcl-2. Thus, theoretically, xenon might be expected to have less neuroapoptogenic potential compared to other anesthetic drugs; indeed, if combined with another anesthetic drug, xenon might be able to counteract (to at least some degree) the apoptogenic properties of the other anesthetic drug.

The inventor herein tested that hypothesis, and found that: (1) xenon has relatively weak neuroapoptogenic potential, in immature animals, when administered by itself, and, (2) when combined with isoflurane, xenon markedly reduces the amount of apoptosis induced by the isoflurane. These results are described in Example 4, below. Accordingly, drug combinations comprising xenon along with another anesthetic agent can offer sufficient anesthesia for surgical purposes, while minimizing the risks of neuro-apoptosis.

Xenon also has been shown recently (Ma et al 2005) to protect against hypoxic and/or ischemic neurodegeneration in the 7 day old rat brain, if administered 4 hours ahead of time, as a preconditioning stimulus. One plausible interpretation of these findings is that xenon may have caused an up-regulation of the BDNF-ERK-CREB pathway, in a manner that shifted the balance of apoptotic proteins versus pro-survival proteins in the survival direction, so that the immature neurons were fortified against a subsequent hypoxic-ischemic insult, and were less prone to die by apoptosis in the aftermath of that insult. This suggests that xenon may have similar anti-apoptosis effects if used to treat traumatic brain injury, or for anesthesia or anti-convulsant purposes, since those conditions involve inactivation of the ERK-CREB pathway, which can be corrected by the activating influence of xenon.

In the case of brain trauma or an ischemic or hypoxic crisis, since the delayed apoptosis reaction usually does not occur until roughly 8 to 24 hours after a traumatic injury or crisis, lithium, xenon, or other anti-apoptotic agents can be administered immediately when a patient arrives in the hospital, allowing as much time as possible for the protective agent to exert its effects and establish a neuroprotective condition, before the full impacts of an apoptotic cascade emerge and begin killing large numbers of neurons in the brain of the patient.

### The PI3Kinase/AKT System as a Target for Modification

The phospho-inositide-3 kinase (PI3K) system is a tyrosine kinase enzyme system that phosphorylates (and thereby activates) various protein molecules. The PI3K system is coupled to NMDA receptors in such a manner that when NMDA receptors are activated, the PI3K system is also activated, which in turn activates a system referred to as the AKT cascade. The AKT cascade sometimes feeds into the ERK cascade, but it also can act independently, through several other pathways, to influence cell survival, in some cases by direct interaction with the Bcl multidomain family of pro- and anti-apoptosis proteins. Accordingly, stimulation of the PI3K/AKT pathway can promote cell survival, and suppression of this pathway can promote apoptosis.

The inventor herein discovered that within 30 minutes after administration of ethanol to infant mice, there is a marked suppression of the activated isoform of AKT. These results are illustrated in the graph shown in FIG. 3C. Therefore, the PI3K/AKT pathway provides another target for therapeutic intervention, with the goal being to counteract the suppressive actions of anesthetic or similar drugs.

Zhu et al 2001 reported that beta-2 adrenergic agonists, by stimulating beta-2 adrenergic receptors, activate the PI3K/AKT pathway and promote cell survival, whereas agonists that stimulate the beta-1 adrenergic receptor promote apoptosis. Noahiko et al 2003 reported that antagonists of the beta-1 adrenergic receptor have an activating effect on the PI3 K/AKT pathway similar to the effect of beta-2 adrenergic agonists. Therefore, beta-2 adrenergic agonists and beta-1 adrenergic antagonists that can readily penetrate blood-brain barriers (such as the beta-2 agonist clenbuterol, and the beta-1 antagonists metoprolol and betaxolol) offer promising candidate drugs, for protecting immature brains against neuro-apoptosis caused by anesthetic, sedating, or anti-convulsant drugs.

It should be noted that the range and variety of different candidate drugs (including various beta-1 and/or beta-2 adrenergic drugs, various muscarinic agonist drugs, lithium, etc.) that can modify “upstream” signaling pathways that would lead to apoptosis, if not blocked by protective drugs, is also potentially very valuable, especially for treating premature babies and infants that need treatment by sedating, anti-convulsant, or similar drugs over extended spans of time, such as for days, weeks, or months at a time. In particular, nearly any organism will respond in a homeostatic, equilibrium-seeking manner to nearly any type of drug that is administered continuously over a prolonged period of time, causing a gradual diminishing of the effects of the continuous drug administration (these effects are referred to by terms such as habituation, tolerance, etc.). Therefore, the availability of an assortment and variety of different drugs that can prevent neuro-apoptosis in immature brains can enable physicians to “rotate” a patient through a various different protective regimens. This can reduce the amount of time that any specific agent must be used, which is likely to help avoid or minimize serious tolerance or habituating effects, and which also is likely to enable a more normal, natural, and balanced growth and development of the immature patient's brain and nervous system.

### PKA and PKC Signaling Pathways

The protein kinase A (PKA) and protein kinase C (PKC) intracellular signaling pathways are also involved in regulating neuronal survival. Like the ERK system, they achieve this by influencing the balance between pro- and anti-apoptotic proteins in the Bcl multidomain family. As this is being written, it is not clear whether the PKA or PKC signaling pathways are altered by apoptogenic drugs used in pediatric and obstetric medicine; nevertheless, because of their known roles, these pathways offer useful and promising targets for therapeutic intervention, to prevent anesthetic, sedative, or similar drugs from triggering neuro-apoptosis. Activation of the PKA or PKC pathways is expected to have pro-survival effects, and pharmacological agents that are known to activate these pathways offer good candidates for achieving the purposes of this invention.

As mentioned above, the muscarinic cholinergic transmitter system is known to be functionally coupled to the PKA and PKC pathways, and activation of muscarinic receptors increases PKA and PKC activation. Therefore, muscarinic cholinergic agonists (such as pilocarpine, as one example) are believed likely to provide neuroprotective effects against anesthetic, sedative, or similar apoptogenic drugs, in immature brains.

Accordingly, when restated in language suited for patent claims, this invention discloses and claims a method for preventing drugs used in pediatric or obstetric medicine from triggering neuroapoptosis in immature mammalian brains, consisting of coadministering, along with a first therapeutic drug that has been shown to be neuroapoptogenic in in vivo tests using neonatal animals, a second drug that has been shown, in in vivo tests using neonatal animals, to substantially reduce neuroapoptosis induced by said neuroapoptogenic compound.

Several terms and phrases in that claim require attention and clarification. In specific:

(1) Archetype examples of such “first therapeutic drugs that have been shown to be neuroapoptogenic in in vivo tests using neonatal animals” include ketamine (a surgical anesthetic that suppresses activity at NMDA receptors), and isoflurane and propofol (surgical anesthetics that activate GABA receptors). However, those are not the only useful therapeutic agents that can be used to suppress neuronal activity in fetuses, babies, or infants, in ways that pose risks of triggering neuro-apoptosis in immature brains. Instead, a fairly wide range of sedative, anxiolytic, and anti-convulsant drugs are used in obstetric and/or pediatric medicine (anxiolytic drugs merit attention, because they are often used to calm and relax babies or infants before surgery, and during various types of painful or uncomfortable procedures). Accordingly, any drugs used in that manner, on immature brains, merit careful testing in light of the disclosures herein, to evaluate the level of neuro-apoptotic risk that any such drug may pose, in immature patients.

If any such drug (either currently known, or hereafter discovered or commercialized) that is used in obstetric and/or pediatric medicine is discovered to pose a substantial risk of inducing neuro-apoptosis, as indicated by in vivo animal tests, then steps can and should be taken, using the disclosures and teachings, to identify one or more drugs that can “substantially reduce” (described below) either the actual damage, or the likely risks, of immature neuro-apoptosis induced by that drug.

At one level, agents such as lithium, xenon, and muscarinic cholinergic agonists, can be used as disclosed herein to substantially reduce the risks of immature neuro-apoptosis that will be posed by any drug that suppresses neuronal activity in brains that are still undergoing the process of synaptogenesis. Accordingly, such drugs can indeed be used, as disclosed herein, to reduce such risks.

However, it should be recognized that, when small, sensitive and highly vulnerable fetuses, babies, and infants are involved, gentler and milder treatments are likely to be safer, and therefore preferable, so long as they have just enough potency and strength to accomplish a desired goal.

Therefore, rather than treating fetuses, babies, and infants with drugs that are potent and powerful, a preferred approach is to develop gentler and milder selected and paired combinations of therapeutic drugs and protective drugs, wherein each type of drug has no more potency than is needed for its task, when used to treat exceptionally small, fragile, and vulnerable patients.

As an example, if a child has only occasional and relatively rare epileptic seizures, he or she may be prescribed a relatively mild anti-convulsant drug, at a dosage that is deliberately designed to block the majority, but not all, of such seizures. That type of deliberately mild treatment can help the child's nervous system develop in as close to a normal manner as possible, with minimal disruptions and alterations by the anti-convulsant drug. If that anti-convulsant drug has been shown to pose substantial risks of neuro-apoptosis, when tested in in vivo animal tests, then a preferred approach would be to combine that drug with a second protective drug that also is deliberately selected, and dosed, to be relatively mild, and minimally intrusive.

Accordingly, rather than limiting the claims to undeniably potent and powerful protective drugs, the principle that milder can be and often is preferable, when treating neonates and infants, should be kept in mind, in interpreting Claim 1 and its dependent claims.

2. Claim 1 refers to a second drug that can “substantially reduce” neuroapoptosis caused by a therapeutic but potentially apoptogenic compound. That reference to a “substantial” reduction of unwanted damage or risk is tied to an arbitrary (or “benchmark”) standard, which requires a reduction of at least 30%, as measured by one or more objective and quantifiable biochemical indicators of neuro-apoptosis (such as concentrations of activated caspase-3 proteins, as described in Example 5 and in various articles coauthored by Olney, cited herein).

Any reference in the claims to any drug includes any salts thereof, and any “analogs” that also have the clinically relevant activities of the referent drug. In general, in pharmacologic terms, an analog is a molecule that resembles another designated molecule, but which has been modified by one or more substituted or altered chemical groups (as examples, a hydrogen atom or hydroxyl group might be replaced by a halogen atoms, a small alkyl or acyl groups, or various other chemical substituents).

The drugs of this invention can be administered by any conventional and known methods, such as orally, by injection or infusion, etc. Preferred modes for any particular compound will be known to those skilled in the art.

### EXAMPLES

### Example #1

### Pilocarpine Pretreatment Reduces Neuroapoptosis Induced by Isoflurane or Midazolam in Infant Mouse Brain

Agents that suppress neuronal activity, including GABAergic anesthetic agents such as midazolam and isoflurane, trigger neuroapoptosis in the infant mouse brain. If suppression of neuronal activity is a sufficient cause of the neuroapoptosis response, it is possible that agents that increase neuronal activity, such as the muscarinic cholinergic agonist, pilocarpine, might counteract this response. To test this hypothesis, we pretreated P5 infant mice with pilocarpine (20 mg/kg), then 2 hrs later exposed them to either isoflurane (0.75% for 4 hrs) or midazolam (9 mg/kg). Control littermates received saline instead of pilocarpine, then were exposed to the same midazolam or isoflurane treatments. The animals were euthanized 6 hrs after initiation of anesthetic drug exposure and the neuroapoptosis response in the caudate nucleus was evaluated by stereological counting of neuronal profiles showing positive IHC staining for activated caspase 3.

It was found that pilocarpine significantly reduced the neuroapoptotic response to either isoflurane or midazolam in the caudate nucleus. Pilocarpine was protective if given 2 or more hrs prior to anesthetic exposure, but not if given at the same time. This suggests that the protective effect is most likely due to altered intracellular pro-survival signaling, perhaps involving protein synthesis, rather than an action at the plasma membrane level counteracting the hyperpolarizing action of the anesthetic drugs. If the neuroprotective effect is dependent on protein synthesis, this would explain why pilocarpine is effective only if administered 2 hours ahead of time, this being the amount of time required for protein synthesis to occur.

### Example #2

### Lithium Prevents Ethanol from Inducing Neuroapoptosis in the Infant Mouse Brain

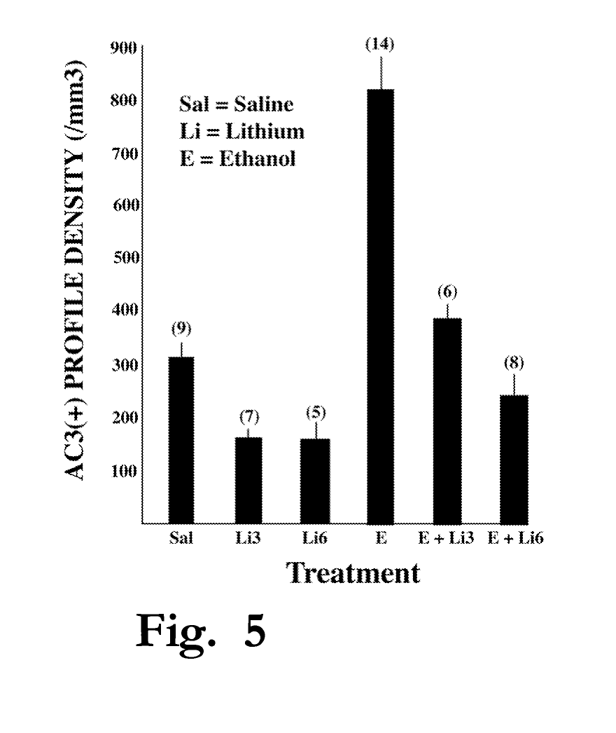

Five day old infant mice were treated either with saline or ethanol (250 mg/g), or lithium carbonate (3 Meq/kg or 6 Meq/kg), or ethanol (250 mg/g) plus lithium (3 or 6 Meq/kg). The animals were euthanized 6 hrs after initiation of drug exposure and the neuroapoptosis response in 5 different brain regions (caudate nucleus, cerebral cortex, thalamus, inferior and superior colliculi, and the cerebellum) was evaluated by stereological counting of neuronal profiles showing positive immunohistochemical staining for activated caspase 3 (AC3). The mean number of AC3-positive profiles per mm³ across all 5 brain regions was calculated for each treatment condition. These values are given in FIG. 5.

Ethanol treatment by itself caused a marked increase in the number of AC3-positive profiles. Lithium treatment by itself caused a marked reduction in the number of AC3-positive profiles compared to saline controls, signifying that lithium suppresses the rate of spontaneous neuroapoptosis that occurs naturally in the developing brain at this age. This effect was equally strong for either dose of lithium (3 or 6 Meq/kg). In animals receiving combined treatment with lithium and ethanol, the lower dose of lithium (3 Meq/kg) brought the apoptosis rate down to the same rate as in saline controls, and the higher dose of lithium brought the rate down to lower than the saline control level. Thus, lithium at either dose provided total protection against ethanol-induced neuroapoptosis. These doses of lithium are in the range that produces blood lithium levels that are considered therapeutic for treating human adults who have manic-depressive illness.

### Example #3

### Lithium Prevents Ketamine or Propofol from Inducing Neuroapoptosis in the Infant Mouse Brain

Ethanol neurotoxicity is mediated through both NMDA and GABA receptors. Having found in Example #2 that lithium protects against the neurotoxicity of ethanol we wanted to determine whether lithium would also protect against the neurotoxicity of a drug, such as ketamine, that acts only through the NMDA receptor, or a drug, such as propofol, that acts only through the GABA receptor. Therefore, two experiments were performed, the first pertaining to ketamine and lithium and the second to propofol and lithium.

### Ketamine and Lithium

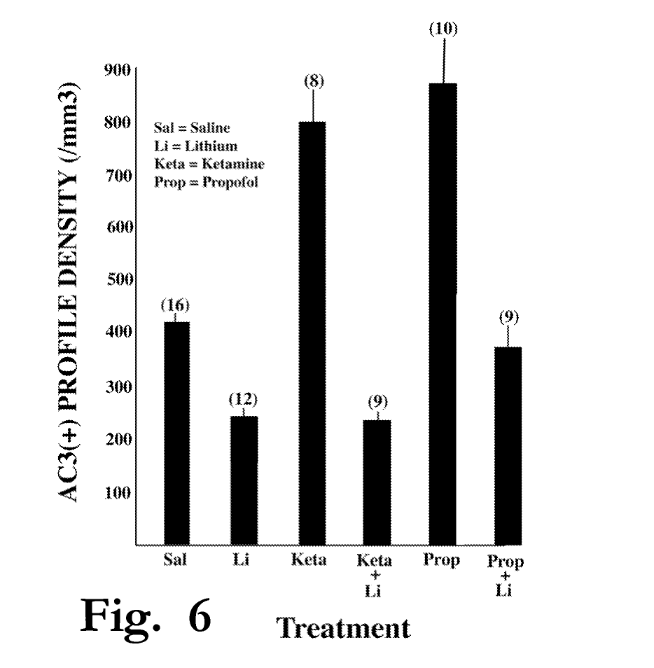

Five day old infant mice were treated either with saline or ketamine (40 mg/kg), or lithium carbonate (6 Meq/kg), or ketamine (40 mg/kg) plus lithium (6 Meq/kg). The animals were euthanized 6 hrs after initiation of drug exposure and the neuroapoptosis response in 2 different brain regions (caudate nucleus, cerebral cortex) was evaluated by stereological counting of neuronal profiles showing positive immunohistochemical staining for activated caspase 3 (AC3). The mean number of AC3-positive profiles per mm³ across these 2 brain regions was calculated for each treatment condition. These values are given in FIG. 6.

Ketamine treatment by itself caused a marked increase in the number of AC3-positive profiles. Lithium treatment by itself caused a marked reduction in the number of AC3-positive profiles compared to saline controls, signifying that lithium suppresses the rate of spontaneous neuroapoptosis that occurs naturally in the developing brain at this age. In animals receiving combined treatment with lithium and ketamine, lithium brought the apoptosis rate down to the same level as for lithium alone, a level substantially lower than the saline control level. Thus, lithium provided total protection against ketamine-induced neuroapoptosis.

### Propofol and Lithium

Five day old infant mice were treated either with saline or propofol (50 mg/kg), or lithium carbonate (6 Meq/kg), or propofol (50 mg/kg), plus lithium (6 Meq/kg). The animals were euthanized 6 hrs after initiation of drug exposure and the neuroapoptosis response in 2 different brain regions (caudate nucleus, cerebral cortex) was evaluated by stereological counting of neuronal profiles showing positive immunohistochemical staining for activated caspase 3 (AC3). The mean number of AC3-positive profiles per mm³ across these 2 brain regions was calculated for each treatment condition. These values are given in FIG. 6.

Propofol treatment by itself caused a marked increase in the number of AC3-positive profiles. Lithium treatment by itself caused a marked reduction in the number of AC3-positive profiles compared to saline controls, signifying that lithium suppresses the rate of spontaneous neuroapoptosis that occurs naturally in the developing brain at this age. In animals receiving combined treatment with lithium and propofol, lithium brought the apoptosis rate down to a level lower than the saline control level. Thus, lithium provided total protection against propool-induced neuroapoptosis.

### Example #4

### Xenon Protects against Isoflurane-Induced Neuroapoptosis in the Infant Mouse Brain

The rare gas, xenon, has anesthetic effects that are mediated by an antagonist action at NMDA glutamate receptors. Isoflurane is a volatile inhalational anesthetic that has been shown to induce neuroapoptosis in the developing mouse brain (see Example #2 above). Experiments were undertaken to determine whether xenon, like other NMDA antagonist drugs, triggers neuroapoptosis in the developing mouse brain, and whether a combination of xenon and isoflurane might cause more or less neuroapoptosis than either drug by itself.

Seven-day-old C57BL/6 mice were exposed to one of four conditions: air (control); 0.75% isoflurane; 70% xenon or 0.75% isoflurane+70% xenon (n≧9 per group) for 4 hours. All pups were euthanized 2 hours later for histopathological evaluation of the brains, using activated caspase-3 immunohistochemical staining to detect apoptotic neurons. Quantitative assessment of the number of apoptotic neurons in caudate/putamen under each condition was performed by unbiased stereology.

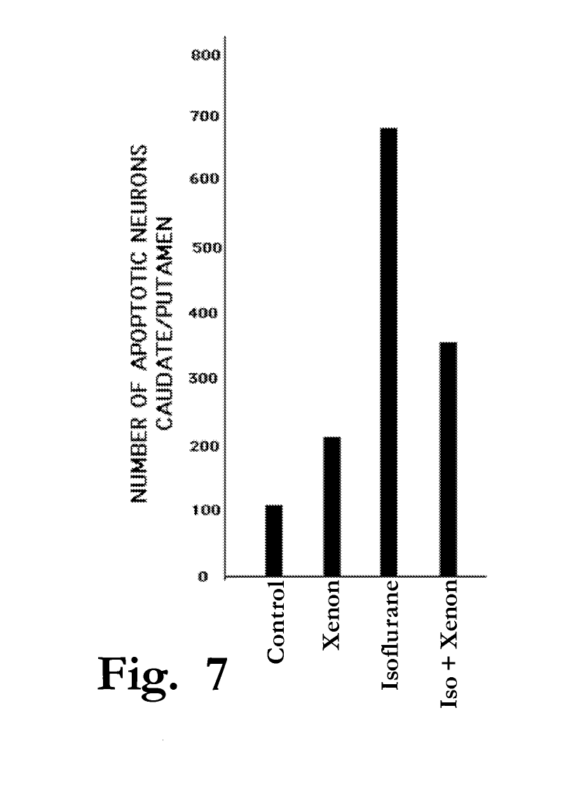

The results are depicted graphically in FIG. 7. In this graph, the number of apoptotic neurons under the control condition is given as 100% and the values for the experimental conditions are given as a percentage of the control. Both xenon alone and isoflurane alone induced a significant increase in neuroapoptosis in the caudate/putamen compared to controls. The neuroapoptotic response to isoflurane was substantially more robust than the response to xenon. When xenon was administered together with isoflurane the apoptotic response was reduced to a level only moderately higher that induced by xenon alone. To fully appreciate the degree of neuroprotection afforded by xenon, it must be recognized that based on all available information, it would have been predicted that xenon and isoflurane would have additive neurotoxicity. Accordingly, this drug combination would have been expected to cause a neurotoxic reaction at least 800% greater than the control, whereas it was actually found to be only about 350% greater than control. Thus, xenon triggers neuroapoptosis to a mild degree, and when combined with isoflurane it does not increase isoflurane's neurotoxicity; rather it strongly protects against isoflurane-induced neuroapoptosis.

### Example 5

### Lithium Protects Against Traumatic Brain Injury in Infant Mice

The method used herein for creating a consistent and reproducible head injury, in infant rats and mice, uses an electromagnetic impact device with a moving coil actuator, mounted on the arm of a stereotaxic holder. The actuator is positioned over the skull of an anesthetized animal, and has a blunt tip aimed at a specific location and angle on the skull. The actuator is driven electromagnetically, and impacts the skull at a computer-controlled speed, delivering a precise concussive force to a targeted zone on the skull.

As described in reports such as Bayly et al 2006, this type of concussive force can be applied to a single side of the head, creating nerve cell death by two different mechanisms. Brain cells directly beneath the skull at the site of impact die by an excitotoxic mechanism, which is confined to the local impact zone, and which leads to end-stage cell death within 4 hours.

Beginning at about 8 hours following injury, a wave of apoptotic neurodegeneration commences, affecting neurons in remote regions of the brain, including the retrosplenial cortex, and the subiculum. During the 16 to 24 hour period after injury, another apoptotic wave commences, affecting neurons in the anterior thalamic nuclei. At about 36 hours after injury, a fourth wave of apoptotic damage commences, causing neurons in the mammillary nuclei to degenerate. Cumulatively, the delayed waves of apoptotic cell death destroy a much larger number of neurons than the acute excitotoxic process; however, the relatively long delays between the time of trauma, and the successive waves of apoptotic cell death, provide a wide window of opportunity, for therapeutic drug intervention to prevent the apoptotic damage.

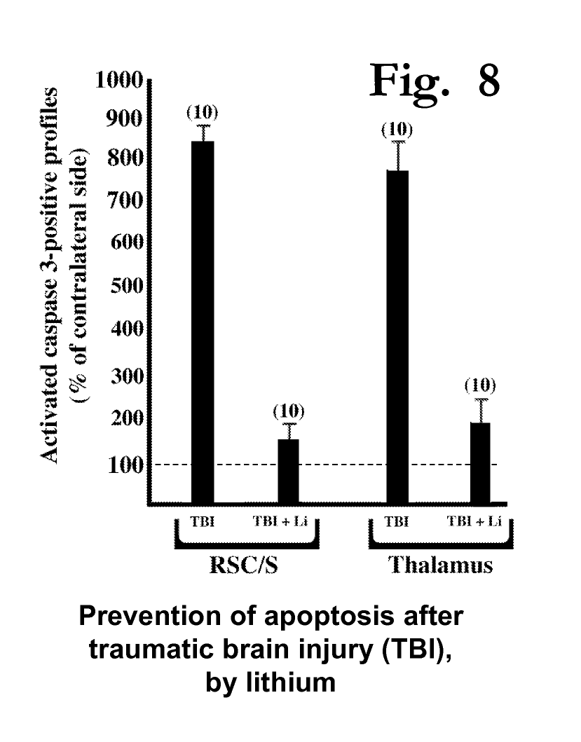

The inventor herein has found that administration of a single dose of a lithium salt (lithium carbonate was used in the tests described herein) to infant mice, either before brain injury, or up to 4 hours after injury, prevented almost all of the apoptotic neurodegeneration. These findings are illustrated in FIG. 8, which shows that in the retrosplenial cortex and subiculum (designated RSC/S), and in the thalamus, 7-fold to 8-fold increases in the number of apoptotic neurons were measured, at 16 hours following traumatic brain injury (TBI), in control animals that were not treated with lithium. However, if lithium was administered 4 hours after the injury, the number of apoptotic neurons was reduced, almost to a “baseline” level that occurs in healthy and uninjured animals.

In these tests, apoptotic neurons were detected by immunohistochemical staining of activated caspase-3 proteins. The quantitative counts were performed in RSC/S and thalamus tissues taken from both (i) the ipsilateral side of the brain, which was damaged, and (ii) the contralateral side of the brain, which was not damaged. Damage levels are presented, in the vertical scale of FIG. 8, as a ratio of ipsilateral (injured tissue) caspase-3 levels, compared to contralateral (uninjured) caspase-3 levels. In that comparison, contralateral counts (representing the undamaged control condition) for tissue samples from each animal were used to determine a normal, 100% (i.e., undamaged baseline) level, and counts from the ipsilateral side were then compared against that baseline number, for that animal.

As can be seen from the graphs, apoptotic neuronal death rates increased to roughly 800% higher than normal, in injured brain tissue from animals that were not treated by lithium. Those damage levels were greatly reduced, almost to the uninjured baseline levels, when lithium was administered at 4 hours after the injury.

### REFERENCES

Bayly P V, Dikranian K T, Black E E, Young C, Qin Y Q, Labruyere J, Olney J W. Spatiotemporal evolution of apoptotic neurodegeneration following traumatic injury to the developing rat brain. Brain Research doi:10.1016/j.brainres.2006.05.102
Dikranian K, Ishimaru M J, Tenkova T, Labruyere J, Qin Y Q, Ikonomidou C, and Olney J W Apoptosis in the in vivo mammalian forebrain. Neurobiol. Dis. 8: 359-379, 2001.
Dobbing J, Sands J The brain growth spurt in various mammalian species. Early Hum Dev, 3:79-84, 1979.
Einat H, Manji H K, Gould T D Du J, Chen G. Possible involvement of the ERK signaling cascade in bipolar disorder: behavioral leads from the study of mutant mice. Drug News Perspect. 16:453-463, 2003.
Naohiko Kobayashi; Shin-ichiro Mita; Kohtaro Yoshida; Takeaki Honda; Tsutomu Kobayashi; Kazuyoshi Hara; Shigefumi Nakano; Yusuke Tsubokou; Hiroaki Matsuoka Celiprolol Activates eNOS Through the PI3K-Akt Pathway and Inhibits VCAM-1 Via NF-B Induced by Oxidative Stress. Hypertension, 42:1004-1009, 2003.
Franks N P. Dickinson R. de Sousa S L. Hall A C. Lieb W R. How does xenon produce anaesthesia?. Nature. 396(6709):324, 1998 Nov. 26.
Ikonomidou, C., Bittigau, P., Ishimaru, M. J, Wozniak, D. F., Koch, C., Genz, K., Price, M. T., Stefovska, V., Horster, F., Tenkova, T., Dikranian, K., & Olney, J. W. Ethanol-induced apoptotic neurodegeneration and fetal alcohol syndrome. Science 287, 1056-1060, 2000.
Ishimaru M J, Ikonomidou C, Tenkova T I, Der T C, Dikranian K, Sesma M A and Olney J W. Distinguishing excitotoxic from apoptotic neurodegeneration in the developing rat brain. J Comp Neurol 408, 461-476, 1999.
Jevtovic-Todorovic V, Hartman R E, Izumi Y, Benshoff N D, Dikranian K, Zorumski C F, Olney J W and Wozniak D F. Early exposure to common anesthetic agents causes widespread neurodegeneration in the developing rat brain and persistent learning deficits. J. Neurosci, 23: 876-882, 2003.
Kerr, J. F. R., A. H. Wyllie, and A. R. Currie Apoptosis: a basic biological phenomenon with wideranging implications in tissue kinetics. British J. Cancer 26: 239-257, 1972.
Ma D, Hossain M, Pettet G K, Luo Y, Lim T, Akimov S, Sanders R D, Franks N P, Maze M. Xenon preconditioning reduces brain damage from neonatal asphyxia in rats. J Cereb Blood Flow Metab. Jul. 20, 2005 (Epub ahead of print).
Olney J W and Ishimaru M J. Excitotoxic Cell Death. In: Cell Death and Diseases of the Nervous System. Koliatsos V E, Ratan R R (Eds), Humana Press Inc, Totowa, N.J., 1999, pp 197-219.
Olney, J. W., Tenkova, T., Dikranian, K., Labruyere, J., Qin, Q. Y., & Ikonomidou, C. Ethanol-induced apoptotic neurodegeneration in the developing C57BL/6 mouse brain. Dev. Brain Res. 133, 115-126, 2002a.
Olney, J. W., Tenkova, T., Dikranian, K., Muglia, L. J., Jermakowicz, W. J., D'Sa, C., & Roth, K. A. Ethanol-induced caspase-3 activation in the in vivo developing mouse brain. Neurobiol. Dis. 9, 205-219, 2002b.
Olney J W. Excitotoxicity, Apoptosis and Neuropsychiatric Disorders. Current Opinion in Pharmacology, 3:101-109, 2003.
Olney J W, Young C, Wozniak D F, Jevtovic-Todorovic V and Ikonomidou C. Do pediatric drugs cause developing neurons to commit suicide? Trends in Pharmacological Sciences (TiPS). 25(3):135-139, 2004.
Olney J W, Young C, Wozniak D F, Ikonomidou C., Jevtovic-Todorovic V Anesthesia-induced developmental neuroapoptosis: Does it happen in humans? Anesthesiology, 101, 273-275, 2004.
Young C, Klocke J, Tenkova T, Choi J, Labruyere J, Qin Y, Holtzman D M, Roth K A, Olney J W. Ethanol-Induced Neuronal Apoptosis in the in vivo Developing Mouse Brain is BAX Dependent. Cell Death and Differentiation. 10: 1148-1155, 2003.
Young C, Tenkova T, Dikranian K, Labruyere J and Olney J W. Excitotoxic versus apoptotic mechanisms of neuronal cell death in perinatal hypoxia/ischemia. Current Molecular Medicine, 4:73-81, 2004.
Young C, Roth K A, Klocke B J, West T, Holtzman D M, Labruyere J, Qin Y-Q, Dikranian K, Olney J W. Role of caspase-3 in ethanol-induced developmental neurodegeneration. Neurobiol Dis. 20:608-614, 2005.
Zhu W Z, Zheng M, Koch W J, Lefkowitz R J, Kobilka B K, Xiao R P. Dual modulation of cell survival and cell death by beta(2)-adrenergic signaling in adult mouse cardiac myocytes. Proc Natl Acad Sci USA. 98:1607-12, 2001.

1. A method for preventing drugs used in pediatric or obstetric medicine from triggering neuroapoptosis in immature mammalian brains, consisting of coadministering, along with a first therapeutic drug that has been shown to be neuroapoptogenic in in vivo tests using neonatal animals, a second drug that has been shown, in in vivo tests using neonatal animals, to substantially reduce neuroapoptosis induced by said neuroapoptogenic compound.

2. The method of claim 1 wherein the first therapeutic drug is selected from the group consisting of anesthetic, sedative, anxiolytic, and anticonvulsant drugs.

3. The method of claim 2 wherein the first therapeutic drug comprises a drug that suppresses activity at the NMDA subclass of glutamate receptors.

4. The method of claim 3 wherein the first therapeutic drug is selected from the group consisting of ketamine, nitrous oxide and xenon.

5. The method of claim 2 wherein the first therapeutic drug comprises a drug that promotes inhibitory transmission through at least one class of GABA receptors.

6. The method of claim 5 wherein the first therapeutic drug is selected from the group consisting of propofol, barbiturates, benzodiazepines, and volatile anesthetics, and analogs thereof that promote inhibitory transmission through at least one class of GABA receptors.

7. The method of claim 2 wherein the first therapeutic drug comprises a drug that suppresses ion flow through at least one class of sodium ion channels in neuronal membranes.

8. The method of claim 4 wherein the first therapeutic drug is selected from the group consisting of phenyloin and valproic acid and analogs thereof that suppress ion flow through at least one class of sodium ion channels in neuronal membranes.

9. The method of claim 1 wherein the first therapeutic drug comprises a drug that suppresses release of glutamate by at least one class of central nervous system neurons.

10. The method of claim 9 wherein the first therapeutic drug is selected from the group consisting of topiramate, lamotrigine, and riluzole, and analogs thereof that suppress release of glutamate by at least one class of central nervous system neurons.

11. The method of claim 1 wherein the second drug that enters a mammalian central nervous system and stimulates neuronal activity is a cholinergic agent that stimulates activity in at least one type of muscarinic cholinergic receptor.

12. The method of claim 11 wherein the cholinergic agent is selected from the group consisting of pilocarpine, arecholine, and analogs thereof that stimulate activity in at least one type of muscarinic cholinergic receptor.

13. The method of claim 1 wherein the second drug that enters a mammalian central nervous system and stimulates neuronal activity is a drug that interacts with at least one apoptosis signaling pathway in a manner that suppresses Bax protein translocation to mitochondrial membranes.

14. The method of claim 1 wherein the second drug increases phosphorylation of at least one type of ERK-MAP-kinase signaling protein.

15. The method of claim 1 wherein the second drug comprises lithium.

16. The method of claim 1 wherein the second drug increases phosphorylation of at least one type of PI3 kinase signaling protein.

17. The method of claim 1 wherein the second drug is selected from the group consisting of beta 1 adrenergic receptor antagonists and beta 2 adrenergic receptor agonists.

18. The method of claim 1 wherein the second drug increases PKC intracellular signaling activity in a manner that reduces neuroapoptosis.

19. The method of claim 1 wherein the second drug increases phosphorylation of at least one type of PKA signaling protein.

20. The method of claim 1 wherein the second drug increases phosphorylation of at least one type of PKC signaling protein.

21. The method of claim 1 wherein the second drug comprises xenon.

22. A method for providing low-risk anesthesia or anticonvulsant therapy for patients selected from the group consisting of pregnant women, fetuses, babies, infants, and children, comprising the administration of xenon gas.

23. The method of claim 22 wherein the xenon gas is coadministered with a second anesthetic or anticonvulsant agent, wherein xenon is administered at a dosage that suppresses apoptogenic activity of the second anesthetic or anticonvulsant agent in immature brains.

24. A method for minimizing neuroapoptosis in an immature brain following a brain insult, consisting of administering, to an immature patient who has suffered a brain insult, a pharmacological agent that interacts with signaling pathways in immature neurons in a manner that has been shown in animal tests to suppress Bax protein translocation to mitochondrial membranes.

25. The method of claim 24 wherein the pharmacological agent is a cholinergic agonist that penetrates blood brain barriers and stimulates activity in at least one type of muscarinic cholinergic receptor.

26. The method of claim 24 wherein the cholinergic agent is selected from the group consisting of pilocarpine, arecholine, and analogs thereof that stimulate activity in at least one type of muscarinic cholinergic receptor.

27. The method of claim 24 wherein the pharmacological agent is xenon.

28. The method of claim 24 wherein the pharmacological agent is lithium.

29. The method of claim 24, wherein the brain insult is selected from the group consisting of traumatic head injury, ischemic blood deprivation, hypoxic oxygen deprivation, and exposure to ethyl alcohol.

30. A method for minimizing neuroapoptosis in an immature brain following a brain insult, consisting of administering, to an immature patient who has suffered a brain insult, a pharmacological agent that interacts with signaling pathways in immature brains in a manner that has been shown in animal tests to prevent or reduce the caspase-3 activation response in immature neurons.

31. The method of claim 30 wherein the pharmacological agent is a cholinergic agonist that penetrates blood brain barriers and stimulates activity in at least one type of muscarinic cholinergic receptor.

32. The method of claim 30 wherein the cholinergic agent is selected from the group consisting of pilocarpine, arecholine, and analogs thereof that stimulate activity in at least one type of muscarinic cholinergic receptor.

33. The method of claim 30 wherein the pharmacological agent is xenon.

34. The method of claim 30 wherein the pharmacological agent is lithium.
